(19) United States
(12) Patent Application Publication
(10) Pub. No.: US 2022/0110604 A1
(43) Pub. Date: Apr. 14, 2022

(54) METHODS AND APPARATUS FOR SMART BEAM-STEERING

(71) Applicant: Liminal Sciences, Inc., Guilford, CT (US)

(72) Inventors: Kamyar Firouzi, San Jose, CA (US); Yichi Zhang, Sunnyvale, CA (US); Mohammad Moghadamfalahi, San Jose, CA (US); Guillaume David, Palo Alto, CA (US)

(73) Assignee: Liminal Sciences, Inc., Guilford, CT (US)

(21) Appl. No.: 17/501,937

(22) Filed: Oct. 14, 2021

Related U.S. Application Data
(60) Provisional application No. 63/228,569, filed on Aug. 2, 2021.
(60) Provisional application No. 63/094,218, filed on Oct. 20, 2020.
(60) Provisional application No. 63/091,838, filed on Oct. 14, 2020.

(51) Int. Cl. A61B 8/08; A61B 8/00; G06N 20/00

(52) U.S. Cl. CPC A61B 8/0808; A61B 8/488; G06N 20/00; A61B 8/5207; A61B 8/469

(57) ABSTRACT

In some aspects, a method includes forming a beam in a direction relative to a brain of a person, the direction being determined by a machine learning model trained on data from prior signals detected from a brain of one or more persons and, after forming the beam, detecting a signal from a region of interest of the brain of the person.

### CROSS REFERENCE TO RELATED APPLICATIONS

This application claims priority under 35 U.S.C. § 119(e) to U.S. Provisional Application Ser. No. 63/091,838, titled “BRAIN MONITOR,” filed Oct. 14, 2020, U.S. Provisional Application Ser. No. 63/094,218, titled “SMART NONINVASIVE TRANSCRANIAL ULTRASOUND SYSTEM,” filed Oct. 20. 2020, and U.S. Provisional Application Ser. No. 63/228,569, titled “METHODS AND APPARATUS FOR SMART BEAM-STEERING,” filed Aug. 2, 2021, all of which are hereby incorporated by reference in their entireties.

### BACKGROUND

Current state of the art in neuromonitoring and neurocritical care typically relies on transcranial ultrasound which requires a high-end ultrasound scanner or a dedicated transcranial doppler system. Such devices are not easy to use and require an operator who has been specially trained on how to place the probe and identify the right location. Identifying such a location typically involves human observation of ultrasound images to determine a current probing location. This can be difficult due to the subtlety of features in ultrasound images, which can be easy to lose with the naked eye. Furthermore, a full three-dimensional search space is relatively large compared to a typical region of interest, which could result in an unforeseen length of time spent searching for the right location. Similarly, magnetic resonance (MR) techniques are not practical for ease-of-use point-of-care applications, especially for rapid screening in the field or continuous monitoring in hospitals, and the associated costs prohibit them from being accessible in many hospital settings.

### SUMMARY

The inventors have recognized the above shortcomings in the current state of the art and have developed novel techniques and devices to address such deficiencies. In particular, the inventors have developed an Artificial-Intelligence (AI)-assisted ultrasound sensing technique capable of autonomously steering ultrasound beams in the brain in two and three-dimensions.

In some embodiments, the beam-steering may be used to scan and interrogate various regions in the cranium, and assisted with AI, may be used to identify a region of interest, lock onto the region of interest, and conduct measurements, while correcting for movements and drifts from the target.

The beam-steering techniques may be implemented in an acoustic device and used to sense, detect, diagnose, and monitor brain functions and conditions including but not limited to detection of epileptic seizure, intracranial pressure, vasospasm, traumatic brain injury, stroke, mass lesions, and hemorrhage. Acoustic or sound in a broad sense may refer to any physical process that involves propagation of mechanical waves, including acoustic, sound, ultrasound, and elastic waves.

In some embodiments, the beam-steering techniques may utilize sound waves in passive or active form, measuring signatures such as reflection, scattering, transmission, attenuation, modulation, etc. of sound waves at one probe or multiple probes to process information and train itself for improved performance over time.

In some aspects, the inventors have developed a method comprising forming a beam in a direction relative to a brain of a person, the direction being determined by a machine learning model trained on data from prior signals detected from a brain of one or more persons. In some embodiments, after forming the beam, the method comprises detecting a signal from a region of interest of the brain of the person.

In some aspects, the inventors have developed a device wearable by or attached to or implanted within a person, comprising a transducer configured to form a beam in a direction relative to a brain of a person, the direction being determined using a machine learning model trained on data from prior signals detected from a brain of one or more persons. In some embodiments, the device comprises a processor configured to process the signal detected from a region of interest of the brain of the person.

In some aspects, the inventors have developed a method of making a device wearable by or attached to or implanted within a person, comprising providing a transducer configured to form a beam in a direction relative to a brain of a person, the direction being determined using a machine learning model trained on data from prior signals detected from a brain of one or more persons. In some embodiments, the method comprises providing a processor configured to process a signal detected from a region of interest of the brain of the person.

In some aspects, the inventors have developed a method comprising receiving a signal detected from a brain of a person. In some embodiments, the method comprises providing data from the detected signal as input to a machine learning model to obtain an output indicating an existence, location, and/or segmentation of an anatomical structure in the brain.

In some aspects, the inventors have developed a device wearable by or attached to or implanted within a person, comprising a transducer configured to detect a signal from a brain of a person. In some embodiments, the device comprises a processor configured to provide data from the detected signal as input to a machine learning model to a machine learning model to obtain output indicating an existence, location, and/or segmentation of an anatomical structure in the brain.

In some aspects, the inventors have developed a method of making a device wearable by or attached to or implanted within a person, comprising providing a transducer configured to detect a signal from a brain of a person. In some embodiments, the method comprises providing a processor configured to provide data from the detected signal as input to a machine learning model to obtain output indicating an existence, location, and/or segmentation of an anatomical structure in the brain.

In some aspects, the inventors have developed a method, comprising receiving a first signal detected from a brain of a person. In some embodiments, the method comprises determining a position of a region of interest of the brain of the person based on data from the first signal and an estimate position of the region of interest of the brain.

In some aspects, the inventors have developed a device wearable by or attached to or implanted within a person, comprising a transducer configured to detect a first signal from a brain of a person. In some embodiments, the device comprises a processor configured to determine a position of a region of interest of the brain of the person based on data from the first signal and an estimate position of the region of interest of the brain.

In some aspects, the inventors have developed a method of making a device wearable by or attached to or implanted within a person, comprising providing a transducer configured to detect a first signal from a brain of a person. In some embodiments, the method comprises providing a processor configured to determine a position of a region of interest of the brain of the person based on data from the first signal and an estimate position of the region of interest of the brain.

In some aspects, the inventors have developed a method, comprising estimating a shift associated with a signal detected from a brain of a person, wherein the shift is indicative of a change in position from which the signal was detected with respect to a position of a region of interest of the brain of the person.

In some aspects, the inventors have developed a device wearable by or attached to or implanted within a person, comprising a processor configured to estimate a shift associated with a signal detected from a brain of a person, wherein the shift is indicative of a change in position from which the signal was detected with respect to a position of a region of interest of the brain of the person.

In some aspects, the inventors have developed a method of making a device wearable by or attached to or implanted within a person, comprising providing a processor configured to estimate a shift associated with a signal detected from a brain of a person, wherein the shift is indicative of a change in position from which the signal was detected with respect to a position of a region of interest of the brain of the person.

In some aspects, the inventors have developed a device for monitoring and/or treating a brain of a person, comprising a transducer comprising a plurality of transducer elements, wherein at least some of the plurality of transducer elements are configured to generate an ultrasound beam to probe a region of the brain.

In some aspects, the inventors have developed a method for monitoring and/or treating a brain of a person, comprising using at least some of a plurality of transducer elements to generate an ultrasound beam to probe a region of the brain.

### BRIEF DESCRIPTION OF DRAWINGS

Various aspects and embodiments will be described with reference to the following figures. It should be appreciated that the figures are not necessarily drawn to scale. For purposes of clarity, not every component may be labeled in every drawing. In the drawings:

FIG. 1 shows an illustrative Acousto-encephalography (AEG) device, in accordance with some embodiments of the technology described herein.

FIG. 2 shows illustrative arrangements of multiple AEG probes over a patient's head, in accordance with some embodiments of the technology described herein.

FIG. 3 shows illustrative system connectivity for an AEG device, in accordance with some embodiments of the technology described herein.

FIG. 4 shows illustrative system/hardware architecture for an AEG device, in accordance with some embodiments of the technology described herein.

FIG. 5 shows an illustrative capacitive micromachined ultrasonic transduce (CMUT) cell, in accordance with some embodiments of the technology described herein.

FIG. 6 shows a block diagram for a wearable device 600 for autonomous beam steering, according to some embodiments of the technology described herein.

FIG. 7 shows example beamforming techniques, according to some embodiments of the technology described herein.

FIG. 8A shows a flow diagram 800 for a method for autonomous beam-steering, according to some embodiments of the technology described herein,

FIG. 8B shows a flow diagram 810 for a method for detecting, localizing, and/or segmenting a ventricle, according to some embodiments of the technology described herein.

FIG. 8C shows a flow diagram 820 for detecting, localizing, and/or segmenting the circle of Willis, according to some embodiments of the technology described herein.

FIG. 8D shows a flow diagram 830 for a method for localizing a blood vessel, according to some embodiments of the technology described herein.

FIG. 8F, shows a flow diagram 840 for method for locking onto a region of interest, according to some embodiments of the technology described herein.

FIG. 8F shows a flow diagram 850 for a method for estimating a shift due to a drift in hardware, according to some embodiments of the technology described herein.

FIG. 8G shows a flow diagram 860 for a method for estimating a shift associated with the detected signal, according to some embodiments of the technology described herein.

FIG. 9 shows diagrams for example beam-steering techniques, according to some embodiments of the technology described herein.

FIG. 10 shows example data processing pipelines, according to some embodiments of the technology described herein.

FIG. 11A shows an example diagram of the Deep Neural Network (DNN) framework used for estimating the relative positions of two regions in the same image, according to some embodiments of the technology described herein.

FIG. 11B shows an example algorithm for template extraction, according to some embodiments of the technology described herein.

FIG. 12 shows a block diagram for reinforcement-learning based guidance for target locking, according to some embodiments of the technology described herein.

FIG. 13 is a block diagram showing an example algorithm for tracking hardware drifts, according to some embodiments of the technology described herein.

FIG. 14 is a block diagram showing an example algorithm for tracking signal shifts, according to some embodiments of the technology described herein.

FIG. 15A shows an example diagram of ventricles, according to some embodiments of the technology described herein.

FIG. 15B shows a flow diagram of an example system for ventricle detection and segmentation, according to some embodiments of the technology described herein.

FIG. 15C shows an example process and data for brain ventricle segmentation, according to some embodiments of the technology described herein.

FIG. 16A shows an example diagram of the circle of Willis, according to some embodiments of the technology described herein.

FIG. 16B shows a flow diagram 1650 of an example algorithm for circle of Willis segmentation, according to some embodiments of the technology described herein.

FIG. 17A shows a flow diagram 1700 for an example algorithm for estimating the vessel diameter and/or curve, according to some embodiments of the technology described herein.

FIG. 17B shows an example vessel diameter estimation, according to some embodiments of the technology described herein.

FIG. 17C shows an example segmentation of a vessel, according to some embodiments of the technology described herein.

FIG. 18 shows an illustrative flow diagram 1800 for a process for constructing and deploying a machine learning algorithm, in accordance with some embodiments of the technology described herein.

FIG. 19 shows a convolutional neural network that may be used in conjunction with an AEG device, in accordance with some embodiments of the technology described herein.

FIG. 20 shows a block diagram of an illustrative computer system 2000 that may be used in implementing some embodiments of the technology described herein.

### DETAILED DESCRIPTION

The current state of the art in neuromonitoring and neurocritical care relies on ultrasound devices that require a trained operator for correctly placing a probe and identifying the region that is to be monitored or measured. As a result, the techniques are limited to monitoring only those regions that can be easily identified through human observation of ultrasound images. This can be limiting, since the brain includes many small and complex regions that can seem indistinguishable through simple observation of such images. Monitoring and measuring features in those regions may provide key insights that can be used as a basis for making diagnoses of, determining the severity of, or treating certain neurological conditions. However, without the ability to identify or locate such regions, the conventional techniques are limited in this respect.

Accordingly, in some aspects, the inventors have developed techniques for detecting a signal from a region of interest of a brain of a person. The techniques include using a transducer to detect the signal from the region of interest by forming a beam in a direction relative to the brain of the person, where the direction is determined by a machine learning model trained on prior signals detected from the brain of one or more persons. For example, the transducer can be an acoustic/ultrasound transducer (e.g., a device that converts electrical to mechanical energy and vice versa). The transducer can be a piezoelectric transducer, a capacitive micromachined ultrasonic transducer, a piezoelectric micromachined ultrasonic transducer, and/or another suitable transducer, as aspects of the technology described herein are not limited in this respect. The detected signal can be the result of a signal applied to the brain. For example, the transducer may detect a signal that has been applied to brain and reflected, scattered, and/or modulated in an acoustic frequency range, after interacting with the brain. The detected signal can be a passive signal generated by the brain. The region of interest can include any region of the brain of any size.

Identifying a region of interest in the brain can be challenging due to the large search volume of the brain. Conventional techniques include probing different regions of the brain at random, while observing ultrasound images. This can include detecting signals from a small region of the brain, observing an image that results from the signal to determine whether it includes the region of interest, and repeating this process until the region of interest appears in an image. As described above, this trial-and-error process can be time-consuming and challenging due to the subtlety of ultrasound images.

Accordingly, in some aspects, the inventors have developed techniques for initially guiding a beam towards a region of interest. In some aspects, the techniques include receiving a first signal from a brain of a person, and determining a position of the region of interest based on an estimate position of the region of interest and data from the first signal. The techniques can further include transmitting an instruction to the transducer to detect a second signal from the region of interest of the brain based on the determined position. For example, the first signal can be detected from a region of the brain that is different than the region of interest or that includes the region of interest. The first signal can be detected after a transducer forms a first beam or first set of beams (e.g., over a plane, a sequence of planes, and/or over a volume.) In some aspects, the direction for forming the first beam can be random, determined by prior knowledge, or output by a machine learning model. In some aspects, the estimate position may be estimated based on prior knowledge and/or estimated using machine learning techniques, as aspects of the technology described herein are not limited in this respect.

In some aspects, once a device is configured to detect signals from a region of the brain to that includes the region of interest, identifying the region of interest can further include detecting, localizing, and/or segmenting the region of interest. For example, detecting the region of interest can include determining whether the region of interest exists in the brain, which may help to inform a diagnosis of a neurological condition. Localizing the region of interest can include identifying the position of the region of interest with respect to the scanned plane, is sequence of planes, or volume. Such information can help to inform future acquisitions for detecting signals from the region of interest. Segmenting the region of interest can include determining information related to the size of the region of interest, such as volume, diameter, or any other suitable measurement. In some embodiments, due to the variability in size, shape, position, and composition of different regions of the brain, it can be challenging to apply the same techniques to detect, localize, and/or segment different regions of interest.

Accordingly, the inventors have developed techniques for detecting, localizing, and/or segmenting anatomical structures in the brain. The techniques can include receiving a signal detected from a brain of a person and providing data from the detected signal as input to a machine learning model to obtain an output indicating an existence, location, and/or segmentation of an anatomical structure in the brain. For example, the anatomical structure can include a ventricle, at least a portion of the circle of Willis, a blood vessel, musculature, and/or vasculature.

In some embodiments, once a region of interest has been identified, using any suitable technique, it may be desirable to take measurements and/or to monitor the region of interest. Monitoring the region of interest over any period of time may involve focusing on the region of interest (e.g., as opposed to probing other regions of the brain). Furthermore, to ensure accuracy and precision of such monitoring and measurements, it can be important to “lock” onto the region of interest to avoid detecting signals from other regions of the brain. For example, locking onto the region of interest may include focusing on the region of interest to detect signals from the region of interest, as opposed to detecting signals from other regions of the brain. However, the position, shape, and size of features in the brain tend to vary between different people, making it challenging to identify clear boundaries of the region of interest for a particular individual. For example, such variances may occur between people of different ages and people of different genders. As a result, techniques that include focusing on a region of interest based on prior knowledge or based on data acquired from the brains of other people may be associated with some error.

Accordingly, in some aspects, the inventors have developed techniques for detecting a signal from and locking onto a region of interest of the brain. The techniques include receiving a signal detected from a brain of a person and determining a position of the region of interest based on data from the signal and an estimate position of the region of interest. For example, the data can include image data, a quality of the first signal, and/or any other suitable data. The estimate position can be determined based on previous knowledge of the position, based on anatomical structures detected in the brain, based on output of a machine learning model, or by any suitable means, as aspects of the technology described herein are not limited in this respect. Determining the position of the region of interest can include providing the data from the first signal and the estimate position as input to a machine learning model to obtain, as output, the position of the region of interest. Based on the position output by the machine learning model, the method can further include transmitting an instruction to a transducer to detect a signal from the region of interest of the brain. In some embodiments, a signal quality may be improved when detecting the signal from the region of interest.

Inadvertent movement of a subject may cause a probe that is fixed to the subject's head to become dislodged, disrupting monitoring or measurements of the region of interest. Furthermore, a beam formed for detecting a signal from a region of interest could gradually shift with respect to the transducer, or a contact quality may change. In these cases, the device may no longer be configured to detect signals from a region of interest of the brain. Rather, the device could begin to detect signals from other regions of the brain, interrupting the continuous monitoring of features in the region of interest and/or interfering with measurements being obtained of features in the region of interest.

Accordingly, in some aspects, the inventors have developed techniques for estimating a shift associated with a signal detected from a brain of a person. In some aspects, the shift is indicative of a change in position from which the signal was detected with respect to a position of a region of interest of the brain of the person. For example, the shift may be due to a change in position of hardware used for detecting the signal from the region of interest and/or a shift in a beam formed by the transducer for detecting the signal from the region of interest. In some aspects, for detecting a change in position of hardware, the techniques can include analyzing image data and/or pulse-wave (PW) Doppler data associated with the detected signal. In some aspects, for detecting a shift in a beam formed by the transducer, the techniques can include analyzing statistical features of signals detected over time and determining whether a shift corresponds to a physiological change.

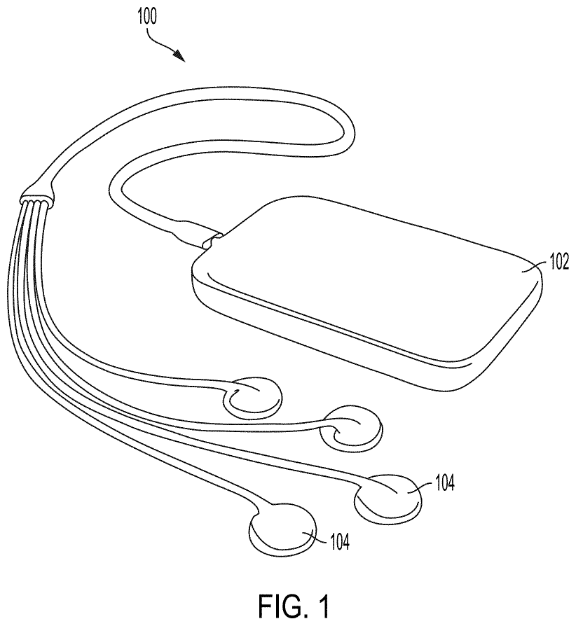

In some embodiments, the beam-steering techniques described herein can be used in conjunction with an acousto-encephalography (or AEG) system, an ultrasound system, and/or any system that passively or actively utilizes sound waves. An exemplary AEG system is described herein, including with respect to FIGS. 1-5.

### Exemplary AEG System

In some aspects, an AEG device described herein can be a smart, noninvasive, transcranial ultrasound platform for measuring brain vitals (e.g., pulse, pressure, flow, softness) that can diagnose and monitor brain conditions and disorders. The AEG device improves over conventional neuromonitoring devices because of features, including but not limited to, being easy-to-use (AEG does not require prior training or a high degree of user intervention) and being smart (AEG is empowered by an AI engine that account for the human factor and as such minimize any errors). It also improves the reliability or accuracy of the measurements. This expands its use cases beyond what is possible with conventional brain monitoring devices. For example, with portable/wearable stick-on probes, the AEG device can be used for both continuous monitoring and/or rapid screening.

In some embodiments, the AEG device is capable of intelligently steering ultrasound beams in the brain in three dimensions (3D). With 3D beam-steering, AEG can scan and interrogate various regions in the cranium, and assisted with AI, it can identify an ideal region of interest. AEG then locks onto the region of interest and conducts measurements, while the AI component keeps correcting for movements and drifts from the target. The AEG device operates through three phases: 1-Lock, 2-Sense, 3-Track.

During Lock, AEG, at a relatively low repetition rate, may “scan” the cranium to identify and lock onto the region of interest, by using AI-based smart beam-steering that utilizes progressive beam-steering to narrow down the field-of-view to a desired target region, by exploiting a combination of various anatomical landmarks and motion in different compartments. Different types of regions of interest may be determined by the “presets” in a web/mobile App such as different arteries or beating at a specific depth in the brain. The region of interest can be a single point, relatively small volume, or multiple points/small volumes at one time. The latter is a unique capability that can probe propagating phenomena in the brain, such as the pulse-wave-velocity (PWV).

During Sense, the AEG device may measure ultrasound footprints of different brain compartments using different pulsation protocols at a much higher repetition rate, to support pulsatile mode, to take the pulse of the brain. The AEG device can also measure continuous wave (CW)-, pulse wave (PW)-, and motion (M)-modes to look at blood flow and motion at select depths.

During Track, the AEG device may utilize a feedback mechanism to evaluate the quality of the measurements. Once the device detects misalignment and misdetection, it goes back to state 1, to properly re-lock onto the target region.

In some embodiments, the AEG device includes core modes of measurements and functionalities, including ability to take the pulse of the brain, ability to measure pulse wave velocity (PWV) by probing multiple regions of interest at one time, and ability to measure other ultrasound modes in the brain, including B-mode (brightness-mode) and C-mode (cross-section mode), blood velocity using CW (continuous-wave) and PW (pulse-wave) doppler, color flow imaging (CFI), PD (power-doppler), M-mode (motion-mode), and blood flow (volume rate).

In some embodiments, the AEG device undertakes a unique approach to estimate intracranial pressure (ICP) based on pulsatility, blood flow, and strain in the brain. The algorithms are built upon a physics-based mathematical model and are augmented with machine learning algorithms. To show the efficacy and train the machine learning algorithm, a clinical study may be performed on a cohort of patients.

In some embodiments, the AEG device can directly measure stiffness in the brain by looking at the time profile of pulsatility and changes in blood flow in the brain. Further, the AEG device can visualize anatomical structures in the brain in 2D and 3D. The AEG device may be equipped with AI for real-time diagnosis of brain health and conditions utilizing vitals in a data-analytics framework to make various diagnoses. The AEG device may use a machine learning model to improve utility and help with critical decision making.

In some embodiments, the AEG is configured to treat the brain of a person using ablation, neuromodulation, ultrasound guided ultrasound (USgUS) treatment, ultrasound guided high intensity focused ultrasound (USgHIFU), and/or drug delivery through a blood brain barrier of the brain. For example, the AEG may be used to directly open the blood brain barrier for drug delivery. In some embodiments, this may include using the AEG to guide an external device for to treatment using the drug delivery through the blood brain barrier.

In some embodiments, AEG, and the techniques described herein, may augment and/or be applicable to systems for brain monitoring and/or treatment using different types of signals, such as acoustic signals, ultrasound imaging, optical imaging, functional near infrared spectroscopy (fNIRS) imaging, computed tomography (CT) imaging, magnetic resonance imaging (MR) imaging, micro-wave and mm-wave sensing and imaging, photoacoustic signals, electroencephalogram (EEG) signals, magnetoencephalogram (MEG) signals, radio frequency (RF) signals, and/or any other suitable signals.

### Form Factor

In some aspects, the AEG device can include a hub and multiple probes to access different brain compartments such as temporal and suboccipital from various points over the head. The hub hosts the main hardware, e.g., analog, mixed, and/or digital electronics. The AEG device can be wearable, portable or an implantable (i.e., under the scalp or skull). In a fully wearable form, the AEG device can also be one or several connected small patch probes. Alternatively, the AEG device can be integrated into a helmet or cap. The AEG device can be wirelessly charged or be wired. It can transfer data wired or wirelessly to a host that can be worn (such as a watch or smart phone), bedside/portable (such as a patient monitor) or implanted (such as a small patch over the neck/arm) and/or to a remote platform (such as a cloud platform). AEG devices may be coupled with acoustic or sound conducting gels (or other materials) or can sense acoustic signals in air (airborne).

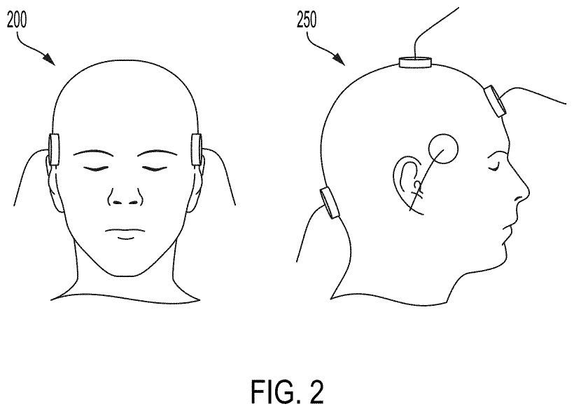

FIG. 1 shows an illustrative AEG device 100 including a hub 102 and multiple probes 104 to access different brain compartments from various points over the head. FIG. 2 shows illustrative arrangements of multiple AEG probes over the head of a patient. For example, in arrangement 200, two probes are placed on the patient's head to access appropriate brain compartments. In another example, in arrangement 250, fives probes are placed around the patient's head to get better access to different compartments of the brain of the person as compared to arrangement 200. The hub may communicate wirelessly with an App or software and/or a cloud platform. The hardware and transducers (or probes) may be designed in a scalable way for future launches of the product or releases of the software, to add new features such as improved algorithms or more sophisticated modes of measurements.

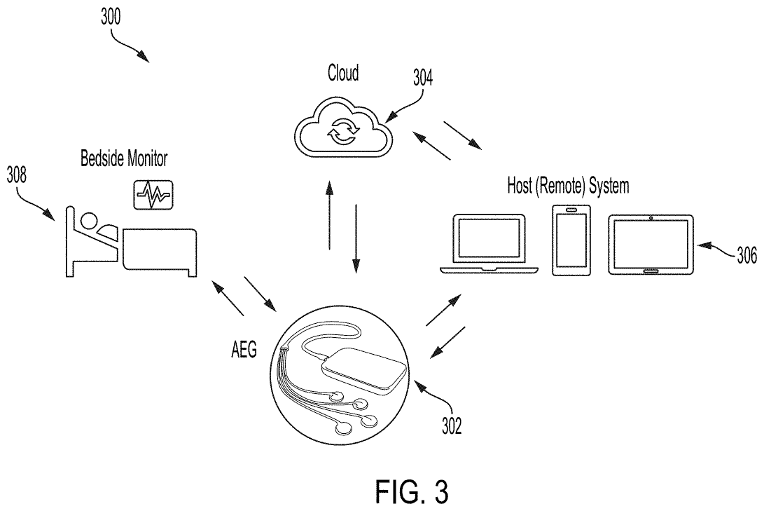

FIG. 3 shows illustrative system connectivity for an AEG device. In block diagram 300, AEG device 302 can be compact and portable/wearable and can continuously stream data to a cloud platform 304 for doctors to view and analyze, equipped with an App or software 306 (on a cell phone, tablet, or a computer) for viewing data and analysis for patient 308. The AEG device can have a wireless hub that is light, portable, and easy to charge. The hub may include a processor to perform part or all the analysis of data from the patient's head. In cases where the hub performs part of the analysis, the remaining analysis may be performed by the cloud platform 304. Such an arrangement may allow for a smaller hub design and/or allow for lower battery or power usage. The AEG device can host additional sensors or probes to provide a comprehensive multimodal assessment, be synced with other instruments and/or be linked to patient monitors. For example, the AEG device can be deployed for at the patient's bedside for remote monitoring. In another example, the AEG device may be capable of communication with a remote system to enable telemedicine applications for analyzing the brain. The AEG device may be capable of continuous monitoring of the brain. For example, the AEG device may be capable of continuous monitoring of the brain for more than six hours, for more than six hours and less than 24 hours, for more than 24 hours, and/or for another time period suitable for continuous monitoring of the brain.

### System/Hardware Architecture

An illustrative system/hardware architecture for an AEG system can include a network of probes for active or passive sensing of brain metrics that are connected to front-end electronics. The front-end electronics may include transmit and receive circuitry, which can include analog and mixed circuit electronics. The front-end electronics can be connected to digital blocks such as programmable logic, a field-programmable gate array (FPGA), processor, and a network of memory blocks and microcontrollers to synchronize, control, and/or pipe data to other subsystems including the front-end and a host system such as a computer, tablet, smartphone, or cloud platform. Programmable logic may provide flexibility in updating the design and functionality over time by updating firmware/software without having to redesign the hardware.

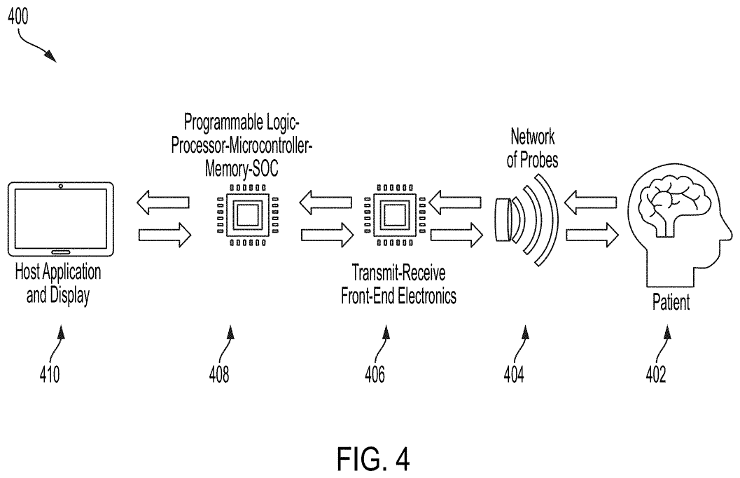

FIG. 4 shows illustrative system/hardware architecture for an AEG device. In block diagram 400, patient 402 may have a network of devices 404, e.g., acoustics transducers, disposed on his or her head. The network of devices 404 may use transmit-receive electronics 406 to transmit data, e.g., wirelessly, BLUETOOTH or another suitable communication means, acquired from the brain and/or skull of patient 402. The transmit-receive electronics 406 can be connected to digital blocks such as programmable logic 408. This data may be processed and/or displayed at display 410. For example, the data may include a waveform or other suitable data received from one or more regions of the patient's brain at an APPLE WATCH or IPHONE or another suitable device that includes display 410.

### Probe (Transducer) Technology

In some aspects, the AEG device includes probes that are acoustic transducers, such as piezoelectric transducers, capacitive micromachined ultrasonic transducers (CMUTs), piezoelectric micromachined ultrasonic transducer (PMUTs), electromagnetic acoustic transducers (EMATs), and other suitable acoustic transducers. Among the feasible techniques for exciting the modes of the skull-brain are direct-surface bonded transducers, wedge transducers, and interdigital transducers/comb transducers. Material and dimensions may determine the bandwidth and sensitivity of the transducer. CMUTs are of particular interest as they can be easily miniaturized even at low frequencies, have superior sensitivity as well as wide bandwidth.

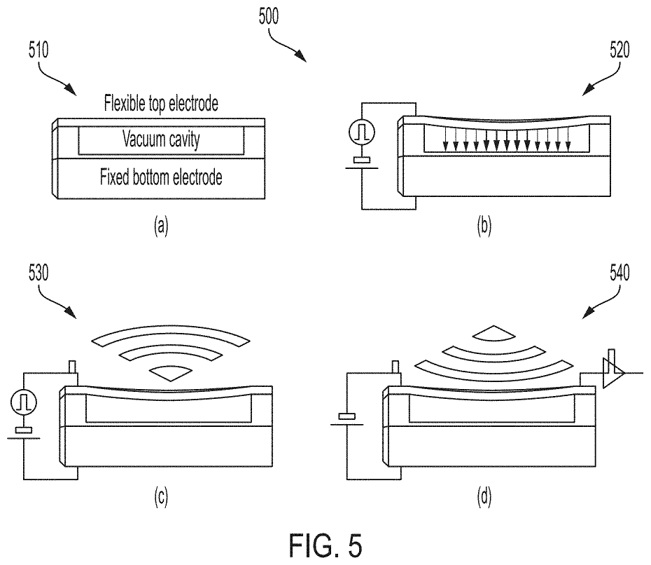

In some embodiments, the CMUT consists of a flexible top plate suspended over a gap, forming a variable capacitor. The displacement of the top plate creates an acoustic pressure in the medium (or vice versa; acoustic pressure in the medium displaces the flexible plate). Transduction is achieved electrostatically, by converting the displacement of the plate to an electric current through modulating the electric field in the gap, in contrast with piezoelectric transducers. The merit of the CMUT derives from having a very large electric field in the cavity of the capacitor, a field of the order of 108 V/m or higher results in an electro-mechanical coupling coefficient that competes with the best piezoelectric materials. The availability of micro-electro-mechanical-systems (MEMS) technologies makes it possible to realize thin vacuum gaps where such high electric fields can be established with relatively low voltages. Thus, viable devices can be realized and even integrated directly on electronic circuits such as complimentary metal-oxide-semiconductor (CMOS). FIG. 5 shows block diagram 500 including illustrations 510, 520, 530, and 540 of a CMUT cell (a) without DC bias voltage, and (b) with DC bias voltage, and principle of operation during (c) transmit and (d) receive.

In some embodiments, a further aspect is collapse mode operation of the CMUT. In this mode of operation, the CMUT cells are designed so that part of the top plate is in physical contact with the substrate, yet electrically isolated with a dielectric, during normal operation. The transmit and receive sensitivities of the CMUT are further enhanced thus providing a superior solution for ultrasound transducers. In short, the CMUT is a high electric field device, and if one can control the high electric field from issues like charging and breakdown, then one has an ultrasound transducer with superior bandwidth and sensitivity, amenable for integration with electronics, manufactured using traditional integrated circuits fabrication technologies with all its advantages, and can be made flexible for wrapping around a cylinder or even over human tissue.

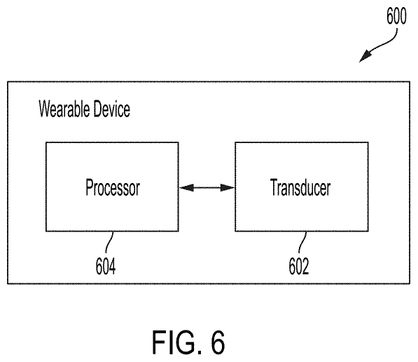

It should be appreciated that the above-described AEG system is an exemplary system with which the smart-beam steering techniques described herein can be used. In particular, the smart-beam steering techniques, described herein including with respect to FIGS. 6-20, can be used in conjunction with any suitable system that passively or actively utilizes sound waves, as aspects of the technology described herein are not limited in this respect.

### Smart-Beam Steering Techniques

### System Overview

In some aspects, the beam-steering techniques described herein can be used to autonomously steer acoustic beams (e.g., ultrasound beams) in the brain. The techniques can be used to identify and lock on regions of interest, such as different tissue types, vasculature, and/or physiological abnormalities, while correcting for movements and drifts from the target. The techniques can further be used to sense, detect, diagnose, and monitor brain functions and conditions, such as epileptic seizure, intracranial pressure, vasospasm, and hemorrhage.

FIG. 6 shows a block diagram for a wearable device 600 for autonomous beam steering, according to some embodiments of the technology described herein. The device 600 is wearable by (or attached to or implanted within) a person. The device 600 includes a transducer 602 and a processor 604.

The transducer 604 may be configured to receive and/or apply to the brain an acoustic signal. In some embodiments, the acoustic signal includes any physical process that involves the propagation of mechanical waves, such as acoustic, sound, ultrasound, and/or elastic waves. In some embodiments, receiving and/or applying to the brain an acoustic signal involves forming a beam and/or utilizing beam-steering techniques, further described herein. In some embodiments, the transducer 604 may be disposed on the head of the person in a non-invasive manner.

The processor 604 may be in communication with the transducer 602. The processor 604 may be programmed to receive, from the transducer 602, the acoustic signal detected from the brain and to transmit an instruction to the transducer 602. In some embodiments, the instruction may indicate a direction for forming a beam for detecting an acoustic signal and/or for applying to the brain an acoustic signal. In some embodiments, the processor 602 may be programmed to analyze data associated with the acoustic signal to detect and/or localize structures and/or motion in the brain, such as different anatomical landmarks, tissue types, musculature, vasculature, blood flow, brain beating, and/or physiological abnormalities. In some embodiments, the processor 602 may be programmed to analyze data associated with the acoustic signal to determine a segmentation of different structures in the brain, such as the segmentation of different tissue types and/or vasculature. In some embodiments, the processor 602 may be programmed to analyze data associated with the acoustic signal to sense and/or monitor brain metrics, such as intracranial pressure, cerebral blood flow, cerebral profusion pressure, and intracranial elastance.

### Beamforming and Beam-Steering

In some embodiments, the transducer e.g., transducer 602) may be configured for transmit- and/or receive-beamforming. The transducer may include transducer elements that are each configured to transmit waves (e.g., acoustic, sound, ultrasound, elastic, etc.) in response to being electrically excited by an input pulse. Transmit beamforming involves phasing (or time-delaying) the input pulses with respect to one another, such that waves transmitted by the elements constructively interfere in space and concentrate the wave energy into a narrow beam in space. Receive-beamforming involves reconstructing a beam by synthetically aligning waves that arrive at and are recorded by the transducer elements with different time delays.

In some embodiments, the functions of a processor (e.g., processor 604) may include generating transmit timing and possible apodization (e.g., weighting, tapering, and shading) during transmit-beamforming, supplying the time delays and signal processing during receive-beamforming, supplying apodization and summing of delayed echoes, and/or additional signal processing-related activities. In some embodiments, it may be desirable to create a narrow and uniform beam with low sidelobes over a long depth. During both transmit and receive operations, appropriate time delays may be supplied to elements of the transducer to accomplish appropriate focusing and steering.

The direction of transmit- and/or receive-beamforming may be changed using beam-techniques. For example, the direction for forming a beam (e.g., beamforming) may be changed by changing the set of time-delays applied to the elements of the transducer. Beam-steering may be performed by any suitable transducer, e.g., transducer 602 to change the direction for forming the beam.

In some embodiments, the beam may be steered in any suitable direction in any suitable order. For example, the beam may be steered left to right, right to left, start at elevation first, and/or start at azimuthal first.

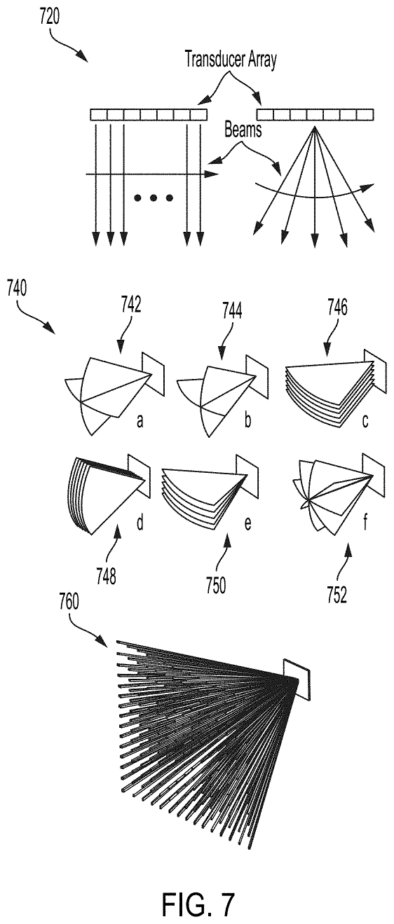

In some embodiments, a transducer consists of multiple transducer elements arranged into an array (e.g., a one-dimensional array or a two-dimensional array). Beam-steering may be conducted by a one-dimensional array over a two-dimensional plane using any suitable architecture. For example, as shown in FIG. 7, a one-dimensional array 720 may include a linear, curvilinear, and/or phased array. Additionally or alternatively, beam-steering may be conducted by a two-dimensional probe array over a three-dimensional volume using any three-dimensional beam-steering technique. For example, as shown in FIG. 7, three-dimensional beam-steering techniques may include planar 740, full volume 760, and random sampling techniques (not shown). Planar beam-steering 740 may include biplane 742, biplane with an angular sweep 744, translational 746, 748, tilt 750, and rotational 752. In some embodiments, three-dimensional beam-steering may be done via mechanical scanning (e.g., motorized holder or robotic arm) and/or fully electronic scanning along the third dimension.

### Smart-Beam Steering System

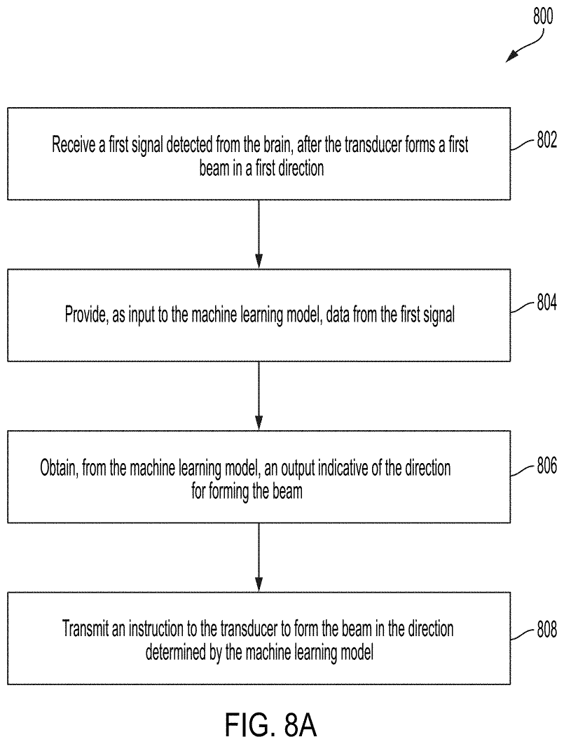

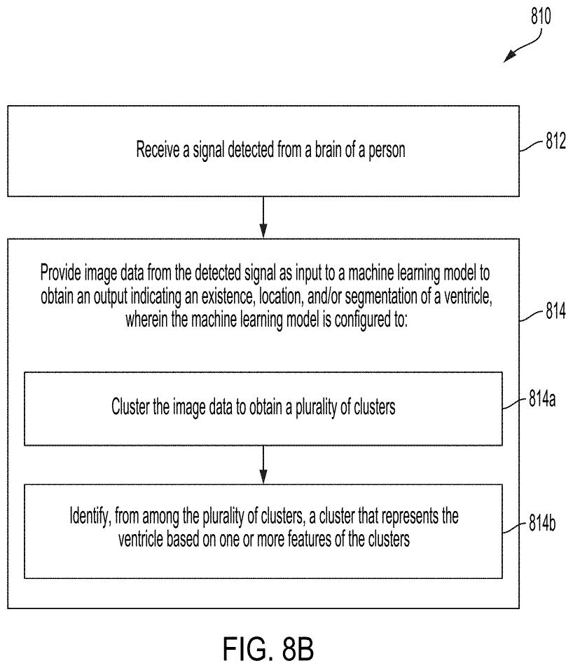

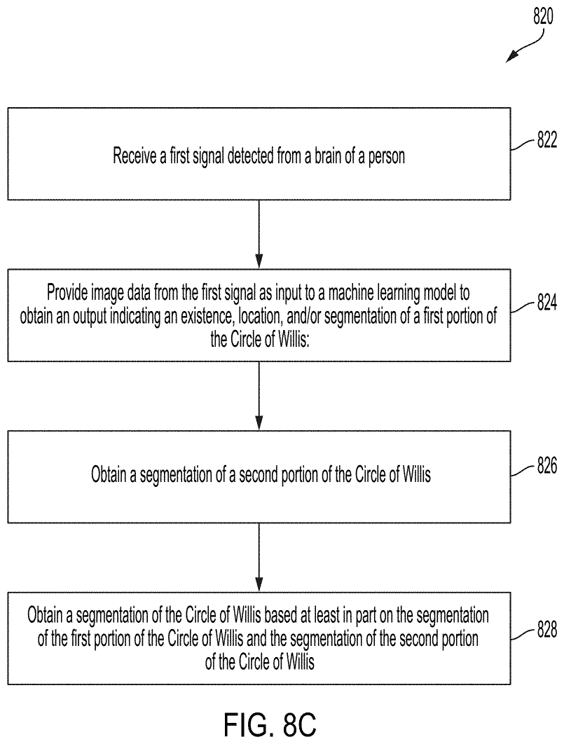

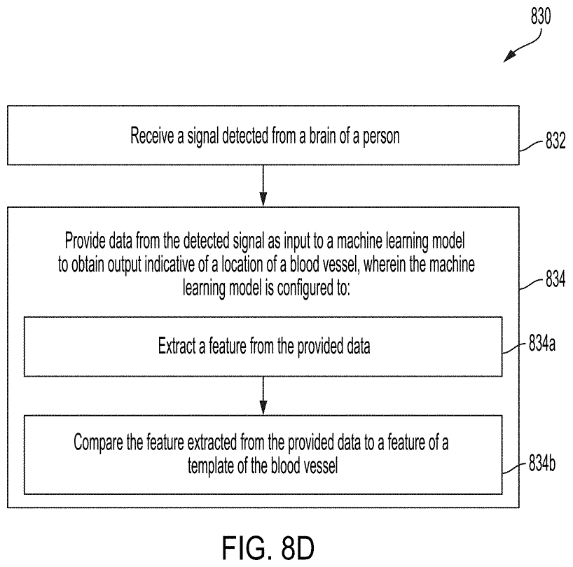

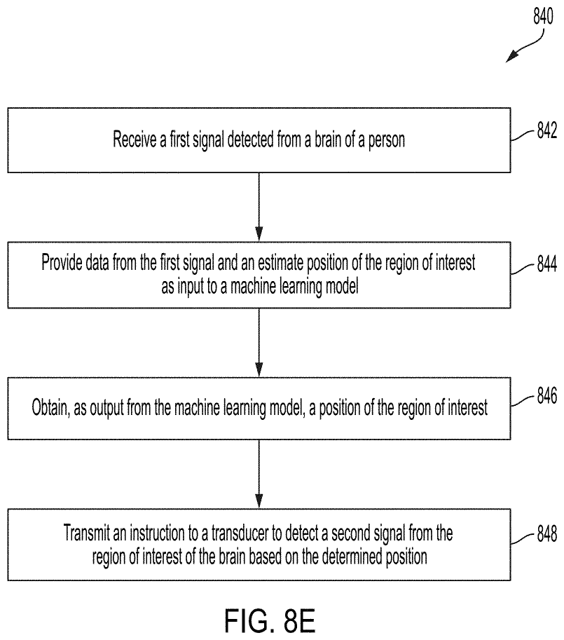

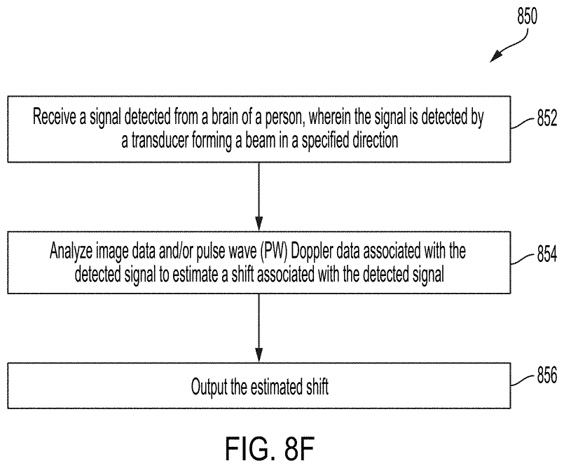

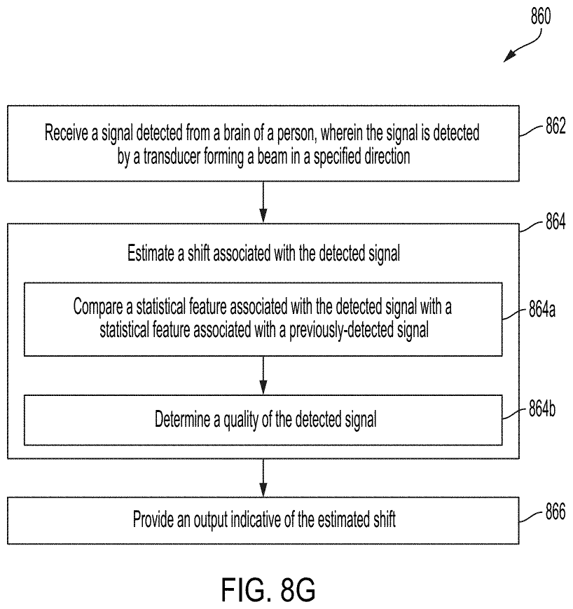

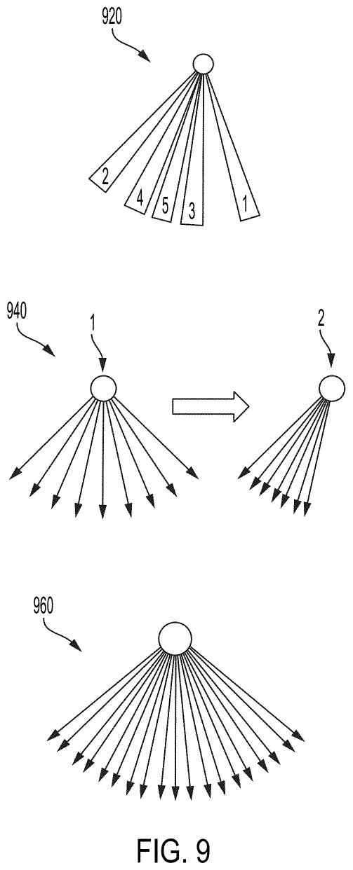

FIG. 8A shows a flow diagram 800 for a method for autonomous beam-steering, according to some embodiments of the technology described herein. In some embodiments, the method may be implemented using a processor, such as processor 604. In some embodiments, the techniques may be used for autonomously detecting a signal from a region of interest of the brain, examples of which are described herein, including at least with respect to FIG. 9.

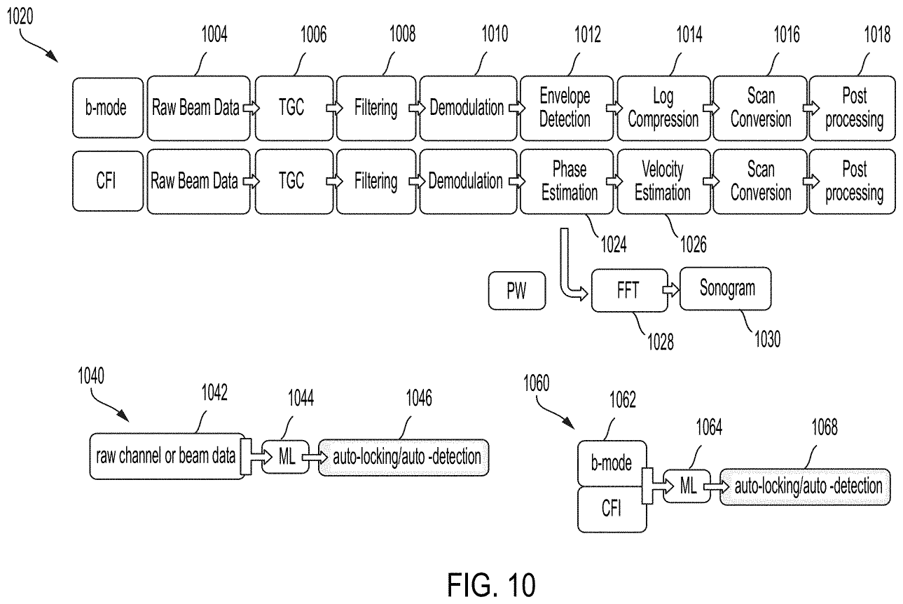

At 802, the techniques include receiving a first signal detected from the brain. In some embodiments, the transducer detects the signal after forming a first beam (e.g., receive- and/or transmit-beamforming) in a first direction. In some embodiments, the first direction may be a default direction, a direction determined using the techniques described herein including with respect to FIG. 9 and/or a direction previously determined using the machine learning techniques described herein. In some embodiments, data from the first signal includes data acquired from a single acoustic beam, a sequence of acoustic beams over a two-dimensional plane, acoustic beams over a sequence of two-dimensional planes, and/or acoustic beams over a three-dimensional volume. In some embodiments, the data may include raw beam data and/or data acquired as a result of one or more processing techniques, such as the processing techniques described herein including with respect to FIG. 10. In some embodiments, the data may be processed to generate B-mode (brightness mode) imaging data, CFI (color-flow imaging) data, PW (pulse-wave) Doppler data, and/or data resulting from any suitable ultrasound modality.

At 804, the techniques include providing the data (e.g., raw data and/or processed data) from the first signal as input to a trained machine learning model. At 806, the trained machine learning model may output the direction, with respect to the brain of a person, for forming the beam to detect the signal from the region of interest.

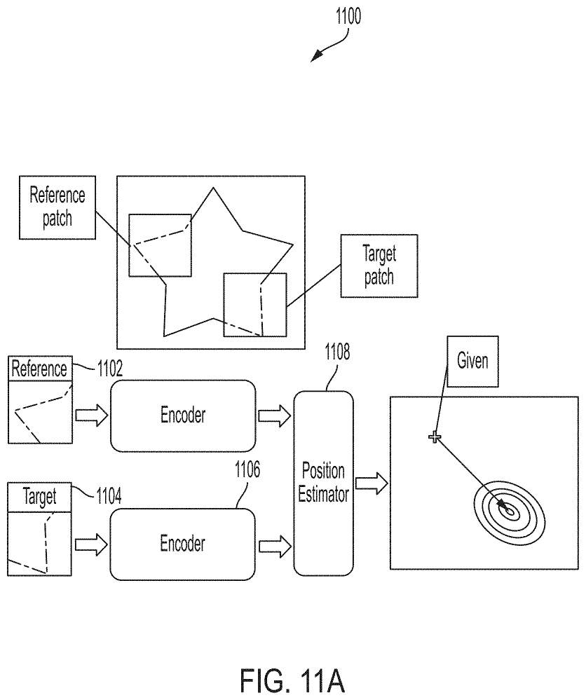

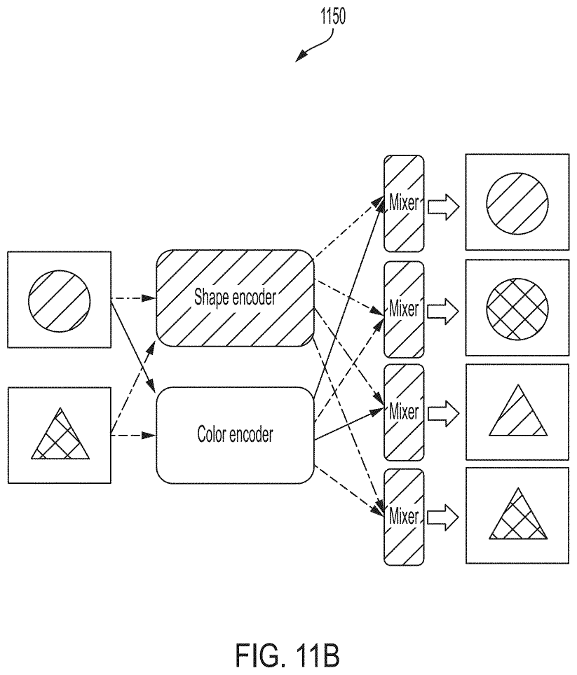

In some embodiments, the trained machine learning model may process the data from the first signal to determine a predicted position of the region of interest relative to the current position (e.g., the position of the region of the brain from which the first signal was detected). In some embodiments, this may include processing the data to detect anatomical landmarks (e.g., ventricles, vasculature, blood vessels, musculature, etc.) and/or motion (e.g., blood flow) in the brain, which may be exploited to determine the predicted position of the region of interest. Based on the predicted position, the machine learning model may determine the direction for forming the second beam and detecting the signal from the region of interest. Machine learning techniques for determining a direction for forming a beam and detecting a signal from the region of interest are described herein including with respect to FIGS. 10 and 11A-B.

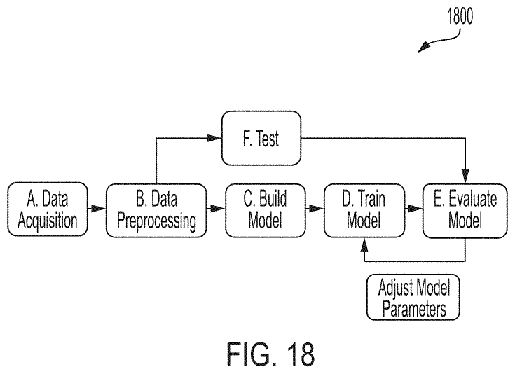

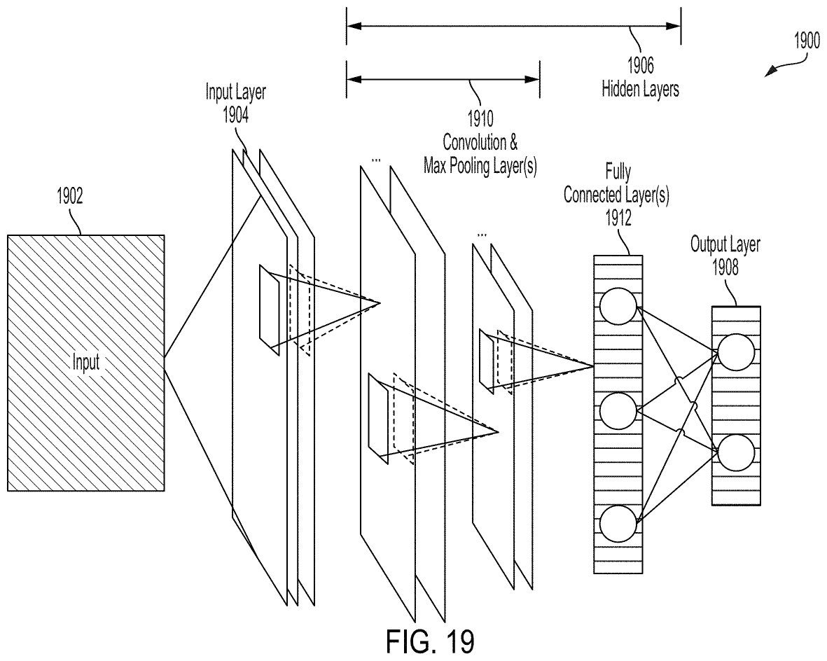

In some embodiments, the machine learning model may be trained on prior signals detected from the brain of one or more persons. The training data may include data generated using machine learning techniques such as Variational Autoencoders (VAE) and Generative Adversarial Networks (GANS) and/or physics based in-silica (e.g., simulation-based) models. An illustrative process for constructing and deploying a machine learning algorithm is described herein including with respect to FIGS. 18-19.

At 806, based on the output from the machine learning model, the processor, e.g., processor 604, transmits an instruction to the transducer to detect the signal from the region of interest by forming a beam in the determined direction. In some embodiments, forming a beam (e.g., transmit- and/or receive-beamforming) in the determined direction may include forming a single beam, forming multiple beams, firming beams over a two-dimensional plane, and/or forming beams over a sequence of two-dimensional planes. In some embodiments, the direction of the beam may include the angle of the beam with respect to the face of the transducer.

In some embodiments, detecting the signal from the region of interest of the brain may include autonomously monitoring the region of interest. This may include, for example, monitoring the region of interest using one or more ultrasound sensing modalities, such as pulsatile-mode (P-mode), continuous wave (CW) Doppler, pulse wave (PW)-Doppler, pulse-wave-velocity (PWV), color-flow imaging (CFI), Power Doppler (PD), and/or motion mode (M-mode). In some embodiments, detecting the signal from the region of interest of the brain may include processing the signal to determine the existence and/or the location of a feature in the brain. For example, this may include determining the existence and/or location of an anatomical abnormality and/or anatomical structure in the brain. In some embodiment, detecting the signal from the region of interest of the brain may include processing the signal to segment a structure in the brain, such as, for example, ventricles, blood vessels and/or musculature. In some embodiments, detecting the signal from the region of interest of the brain may include processing the signal to determine one or more brain metrics, such as an intracranial pressure (ICP), cerebral blood flow (CBF), cerebral profusion pressure (CPP), and/or intracranial elastance (ICE). In sonic embodiments, detecting the signal from the region of interest may correct for beam aberration.

In some embodiments, the region of interest of the brain may include any suitable region(s) of the brain, as aspects of the technology described herein are not limited in this respect. In some embodiments, the region of interest may depend on the intended use of the techniques described herein. For example, for determining a distribution of motion in the brain, a large region of the brain may be defined as the region of interest. As another example, for determining whether there is an embolism in an artery of the brain, a small and precise region may be defined as the region of interest. As yet another example, for measuring blood flow in a blood vessel, two different regions of the brain may be defined as the regions of interest. In some embodiments, an suitable region of any suitable size may be defined as the region of interest, as aspects of the technology are not limited in this respect.

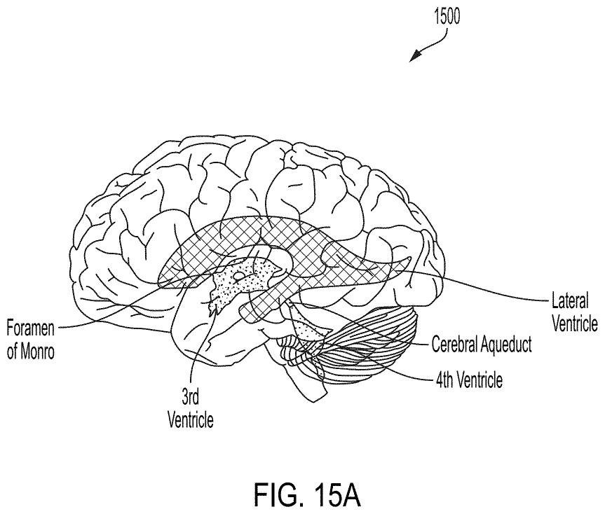

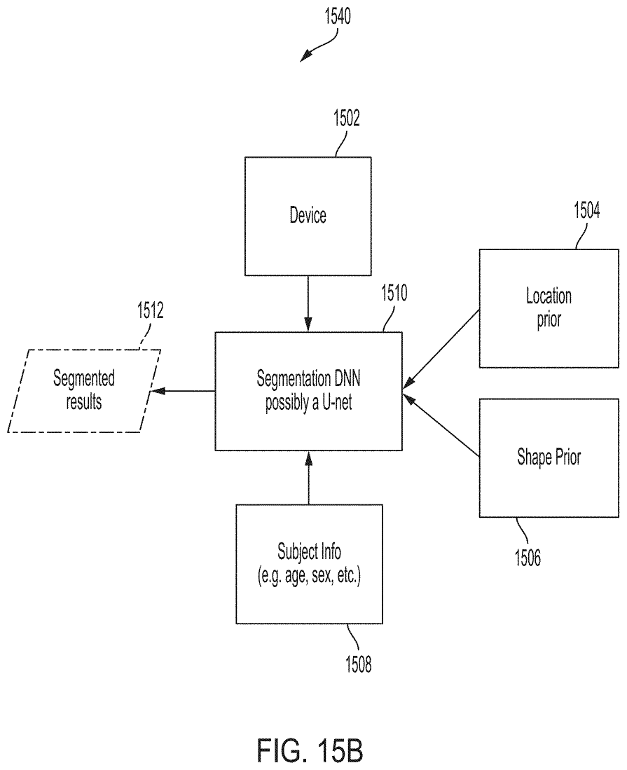

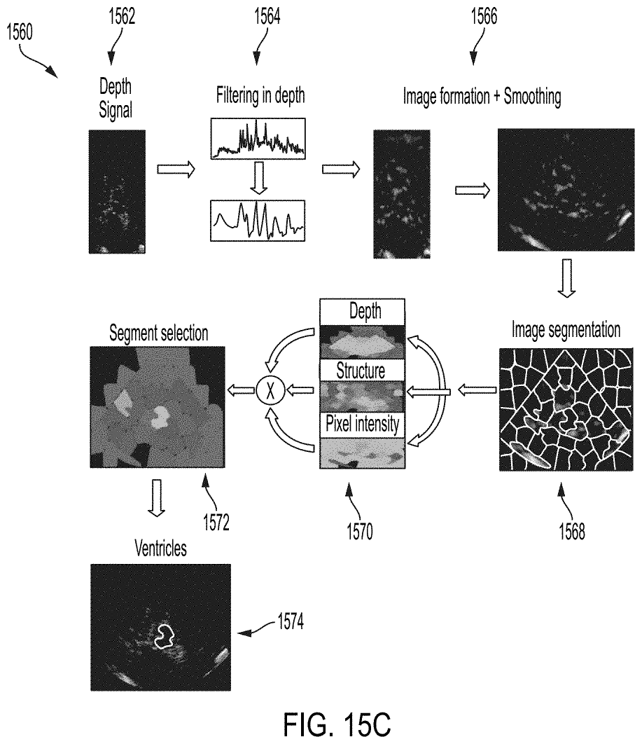

In some embodiments, in identifying a position of a region of interest, the techniques may include detecting, localizing, and/or segmenting anatomical structures in the brain. In addition to aiding in the identification of the region of interest, the results of detection, localization, and segmentation may be useful fur informing diagnoses, determining one or more brain metrics, and/or taking measurements of the anatomical structures. Techniques for detecting, localizing, and/or segmenting anatomical structure in the brain are described herein including with respect to FIGS. 8B-8D. Examples for detecting, localizing, and/or segmenting such structures are described herein including with respect to FIGS. 15A-17C.

FIG. 8B shows a flow diagram 810 for a method for detecting, localizing, and/or segmenting a ventricle, according to some embodiments of the technology described herein. In some embodiments, the method may be implemented using a processor, such as processor 604. Examples for detecting, localizing, and segmenting a ventricle are described herein including with respect to FIGS. 15A-C.

At 812, the techniques include receiving a signal detected from the brain of a person. In some embodiments, the signal may be received from a transducer (e.g., transducer 602) configured to detect a signal from a region of interest. For example, the autonomous beam-steering techniques described herein, including with respect to FIG. 8A, may be used to guide a beam towards the region of interest. As other examples, the direction for forming the beam and detecting the signal from the region of interest may be determined based on prior knowledge, output by a machine learning model, and/or identified by a user.

At 814, data from the detected signal is provided to a machine learning model to obtain an output indicating the existence, location, and/or segmentation of the ventricle. In some embodiments, the data includes image data, such as brightness mode (B-mode) image data.

In some embodiments, the machine learning model may be configured, at 814a, to cluster the image data to obtain a plurality of clusters. For example, the image data may be clustered based on pixel intensity, proximity, and/or using any other suitable techniques as embodiments of the technology described herein are not limited in this respect.

At 814b, the machine learning model is configured to identify, from among the plurality of clusters, a cluster that represents the ventricle. In some embodiments, the cluster may be identified based on one or more features of the clusters. For example, features used for identifying such a cluster may include a pixel intensity, a depth, and/or a shape associated with the cluster. In some aspects, the features associated with a cluster may be compared to a template of the region of interest. For example, the template may define expected features of the cluster that represents the ventricle such as an estimate pixel intensity, depth, and/or shape. The template may be determined based on data obtained from the brains of one more reference subjects. In some aspects, the techniques may include identifying a cluster that has features that to are similar to those of the template.

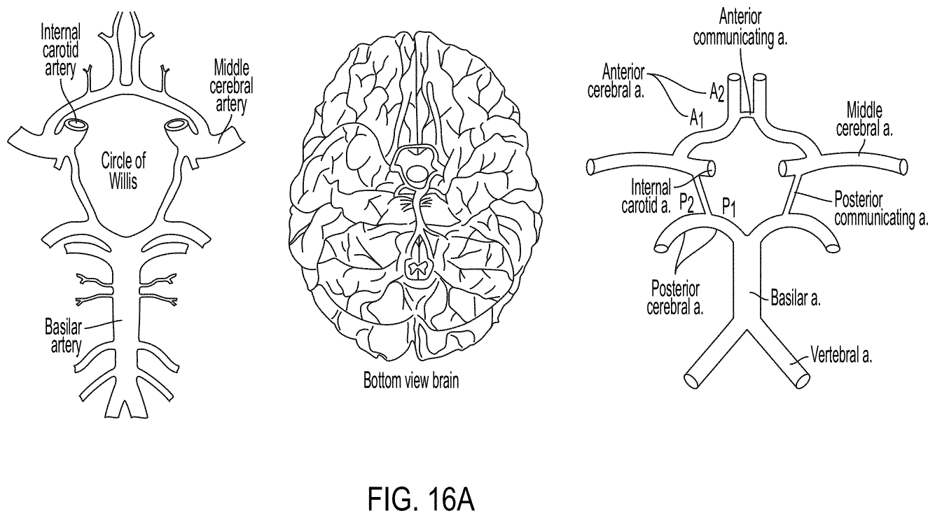

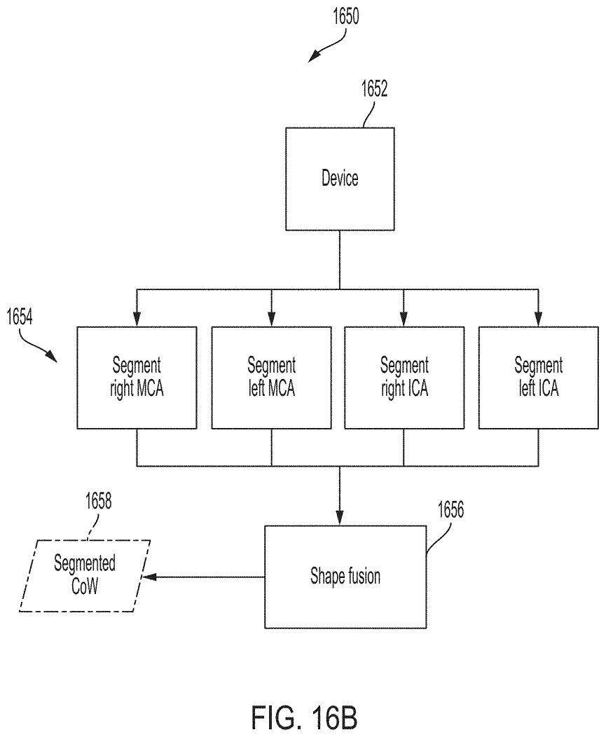

FIG. 8C shows a flow diagram 820 for detecting, localizing, and/or segmenting the circle of Willis, according to some embodiments of the technology described herein. In some embodiments, the techniques may be implemented using a processor, such as processor 604. Examples for detecting, localizing, and segmenting the circle of Willis are described herein including with respect to FIGS. 16A-B.

At 822, the techniques include receiving a first signal detected from the brain of a person. In some embodiments, the first signal may be received from a transducer (e.g., transducer 602) configured to detect a signal from a region of interest. For example, the autonomous beam-steering techniques described herein including with respect to FIG. 8A may be used to guide the beam towards the region of interest. As other examples, the direction for forming the beam and detecting the signal from the region of interest may be determined based on prior knowledge, output by a machine learning model, and/or identified by a user.

At 824, data from the first signal is provided to a machine learning model to obtain an output indicating the existence, location, and/or segmentation of a first portion of the circle of Willis. In some embodiments, the data includes image data, such as, for example, B-mode image data and/or CFI data. In some embodiments, segmenting the first portion of the circle of Willis may include using the techniques described herein including at least with respect to act 814 of flow diagram 810. For example, the machine learning model may be configured to cluster image data and compare features of each cluster to those of a template of the first portion of the circle of Willis.

At 826, the method includes obtaining a segmentation of a second portion of the circle of Willis. In some aspects, the second portion of the circle of Willis may be segmented according to the techniques described herein including with respect to act 824. As a non-limiting example, the first portion of the circle of Willis may include the left middle cerebral artery (MCA), while the second portion of the circle of Willis may include the right internal carotid artery (ICA). Additionally or alternatively, a portion of the circle of Willis may include the right MCA, the left ICA, or any other suitable portion of the circle of Willis, as embodiments of the technology described herein are not limited in this respect.

A segmentation of the circle of Willis may be obtained at 828 based at least in part on the segmentations of the first and second portions of the circle of Willis. For example, obtaining the segmentation of the circle of Willis may include fusing the segmented portions.

In some embodiments, the method 820 includes segmenting the circle of Willis in portions (e.g., the first portion, the second portion, etc.), rather than in its entirety, due to its size and complexity. However, the techniques described herein are not limited in this respect and may be used to segment the whole structure, as opposed to segmenting separate portions before fusing them together.

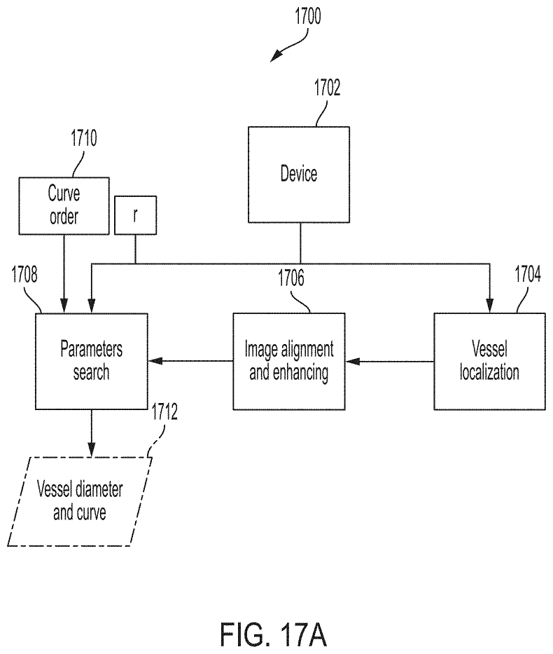

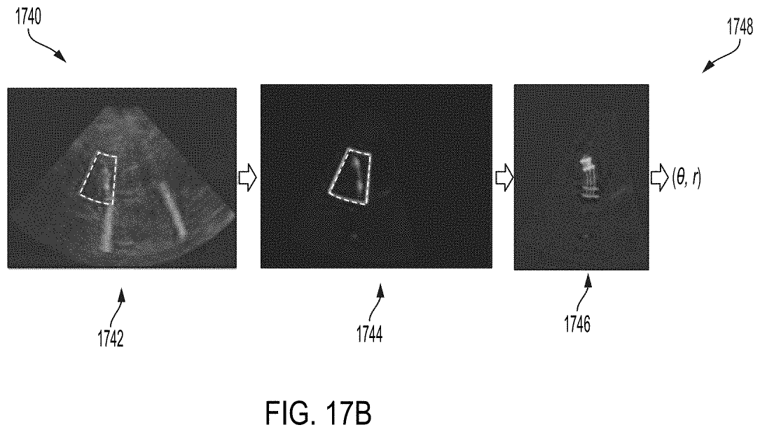

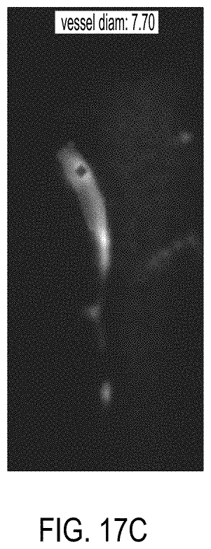

FIG. 8D shows a flow diagram 830 for a method for localizing a blood vessel, according to some embodiments of the technology described herein. For example, in some embodiments, the techniques may be used to localize portions of the circle of Willis since the circle of Willis includes a network of blood vessels. Examples for detecting and localizing a blood vessel are described herein including with respect to FIGS. 17A-C. In some embodiments, the techniques may be implemented using a processor, such as processor 604.

At 832, the techniques include receiving a signal detected from the brain of a person. In some embodiments, the signal may be received from a transducer (e.g., transducer 602) configured to detect a signal from a region of interest. For example, the autonomous beam-steering techniques described herein, including with respect to FIG. 8A, may be used to guide the beam towards the region of interest. As other examples, the direction for forming the beam and detecting the signal from the region of interest may be determined based on prior knowledge, output by a machine learning model, and/or identified by a user.

At 834, data from the detected signal is provided to a machine learning model to obtain an output indicating the location of the blood vessels. In some embodiments, the date comprises image data, such as brightness mode (B-mode) image data and/or color flow image (CFI) image data.

In some embodiments, the machine learning model is configured, at 834a, to extract a feature from the provided data. In some embodiments, an extracted feature may include features that are scale and/or rotation invariant. In some embodiments, the features may be extracted utilizing the middle layers of a pre-trained neural network model, examples of which are provided herein.

At 834b, the extracted features are compared to features extracted from a template of the vessel. In some embodiments, the template may be based on data previously-obtained from the brains of one or more subjects. The results of the comparison may be used to identify the location of the vessel with respect to the image data. In some embodiments, identifying the location based on scale and/or rotation invariant features may help to identify a location with minimal vessel variations. In some embodiments, additional data may be acquired based on the identified location of the vessel (e.g., additional B-mode and/or CFI frames), which may be used for taking subsequent measurements of the vessel and/or blood flow in the vessel.

As described above, features of a region of interest, such as the size, shape, and position, may vary between different people. Thus, it may not be possible to estimate the precise position of the region of interest for each individual based on prior knowledge or training data. For example, the techniques described herein, including with respect to FIGS. 8A and 8B, utilize prior data collected from the brain of subjects in a training population to estimate a position of the region of interest in the subject. However, these techniques may yield only an approximate position of the region of interest. Therefore, the techniques described herein provide for a method for accounting for these subject-dependent variables.

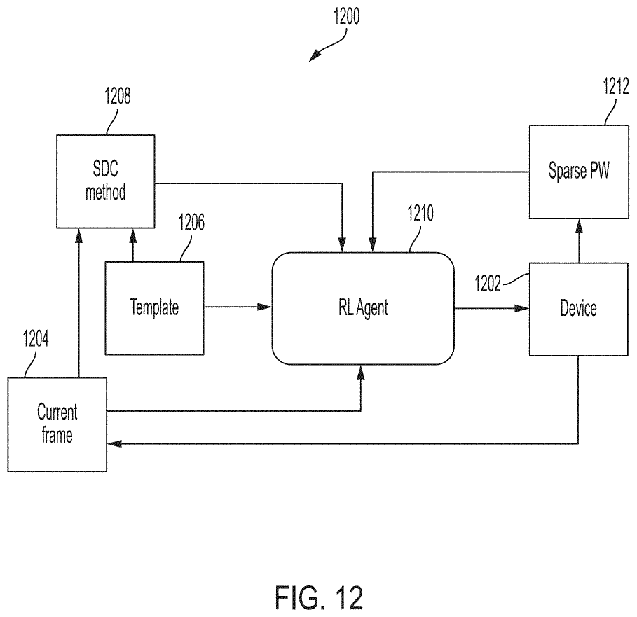

FIG. 8E shows a flow diagram 840 for method for locking onto a region of interest, according to some embodiments of the technology described herein. In some embodiments, the method may be implemented using a processor, such as processor 604. Example techniques for locking onto the region of interest are described herein including with respect to FIG. 12.

At 842, the techniques include receiving a first signal detected from a brain of a person. In some embodiments, the signal may be detected by a transducer (e.g., transducer 602) forming a beam in a specific direction. For example, the direction may be determined by a user, based on output from a machine learning model (e.g., described herein including with respect to FIGS. 8A and B), based on prior knowledge of the direction for forming the bean, or using any other suitable techniques for determining such a direction, as embodiments of the technology are not limited in this respect.

At 844, the data from the first signal, as well as an estimate of a position of a region of interest, are provided as input to a machine learning model. For example, the data from the first signal may include B-mode image data, CH data, PW Doppler data, raw beam data, or any suitable type of data related to the detected signal, as embodiments of the technology are not limited in this respect. In some embodiments, the data from the signal may be indicative of a current region from which the transducer is detecting the signal. The estimate position of the region of interest may be determined based on prior physiological knowledge, prior data collected from the brain of another person or persons, output of a machine learning model, output of techniques described herein including at least with respect to FIGS. 8A-B, data obtained from the detected signal (e.g., the first signal), or determined in any other suitable way as embodiments of the technology are not limited in this respect. In some embodiments, additional information, such as a template of the region of interest may also be provided as input to the machine learning model. For example, a template may provide an estimate position, shape, color, and/or a number of other features estimated for a region of interest.

At 846, a position of the region of interest is obtained as output from the machine learning model. For example, the machine learning model may include any suitable reinforcement-learning technique for determining the position of the region of interest. In some embodiments, the determined position of the region of interest, output by the machine learning model, may be another estimate position of the region of interest (e.g., not the exact position of the regions of interest).

At 848, an instruction is transmitted to a transducer to detect a second signal from the region of interest of the brain based on the determined position of the region of interest. In some embodiments, the instruction includes a direction for forming a beam to detect a signal from the region of interest. For example, the direction may be determined based on the output of the machine learning model (e.g., the position of the region of interest) and/or as part of processing data using the machine learning model. In some embodiments, as described above, the determined position of the region of interest may also be an estimate position of the region of interest. Therefore, the instruction may instruct the transducer to detect the second signal from the estimate position of the region of interest determined by the machine learning model, rather than an exact position of the region of interest. In some embodiments, the quality of the second signal may be an improvement over the quality of the first signal. For example, the second signal may have a higher signal-to-noise ratio (SNR) than that of the first signal.

As described above, after locating and/or locking onto a region of interest, it may be desirable to continue to detect signals from the region of interest. However, over time, a signal may no longer be detected from the desired region. For example, due to patient movement, a stick-on probe may become dislodged or slip from its original position. Additionally or alternatively, the beam may gradually shift with respect to the initial direction in which it was formed. Therefore, the techniques described herein provide for techniques for addressing any hardware and/or beam shifts.

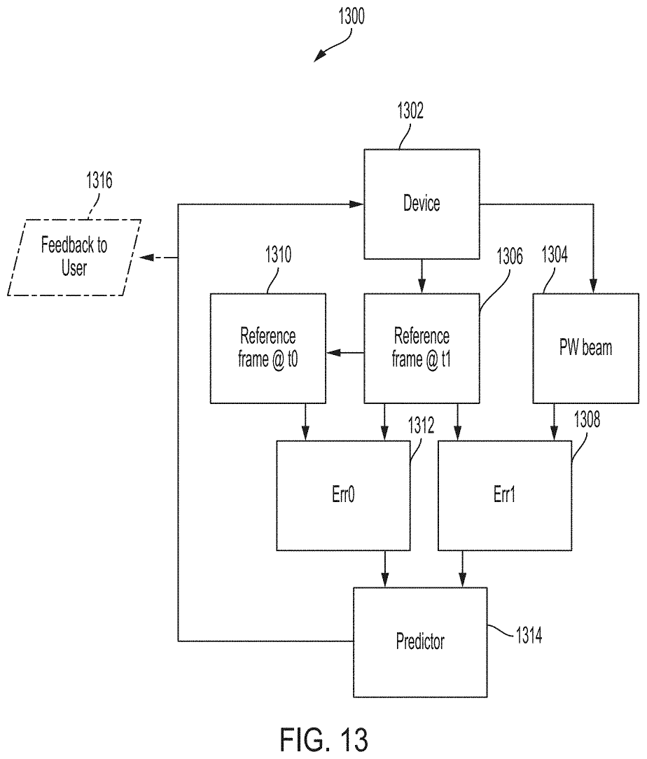

FIG. 8F shows a flow diagram 850 for a method for estimating a shift due to a shift in hardware, according to some embodiments of the technology described herein. In some embodiments, the method may he implemented using a processor, such as processor 604, Example techniques are described herein including with respect to FIG. 13.

At 852, the techniques include receiving a signal detected from a brain of a person. The signal is detected by a transducer (e,g., transducer 602) forming a beam in a specified direction. For example, the direction may be determined by a user, based on output from a machine learning model (e.g., described herein including with respect to FIGS. 8A, 8B, and 8E), based on prior knowledge of the direction for forming the beam, or using any other suitable techniques for determining such a direction, as embodiments of the technology are not limited in this respect.

At 854, the techniques include analyzing image data and/or pulse wave (PW) Doppler data associated with the detected signal to estimate a shift associated with the detected signal. In some embodiments, the techniques may include one or more processing steps to process data associated with the signal to obtain B-mode image data and/or PW Doppler data. In some embodiments, analyzing the image data and/or PW Doppler data may include one or more steps. For example, the image data may be analyzed in conjunction with the PW Doppler data to indicate a current position and/or possible angular beam shifts that occurred during signal detection. Additionally or alternatively, a current image frame may be compared to a previously-acquired image frame to estimate a change in position of the region of interest within the image frames over time.

At 856, the techniques include outputting the estimated shift. For example, the estimated shift may be used as input to a motion prediction and compensation framework, such as a Kalman filter. This may he used to adjust the beam angle to correct for angular shifts, such that the transducer continues to detect signals from a region of interest. Additionally or alternatively, feedback indicative of the estimated shift may be provided through a user interface. For example, based on the feedback, a user may correct for shifts when the hardware does not have the capability.

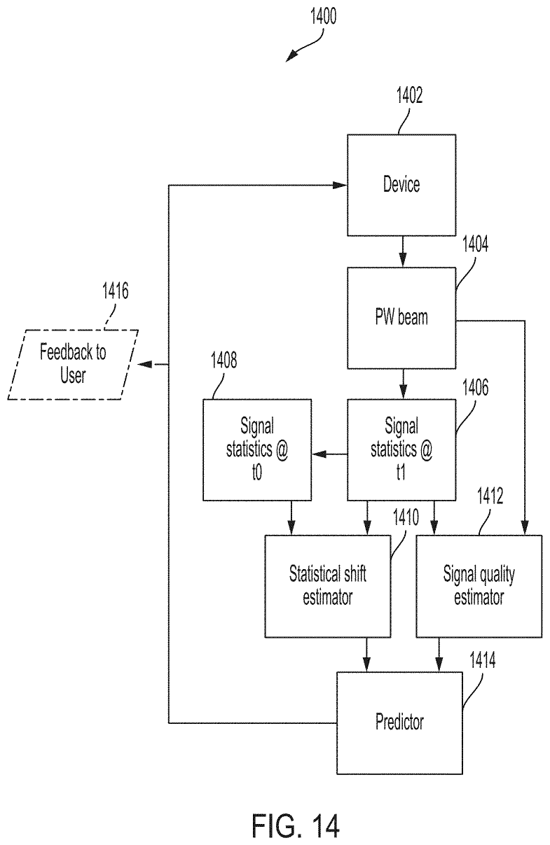

FIG. 8G shows a flow diagram 860 for a method for estimating a shift associated with the beam, according to some embodiments of the technology described herein. In some embodiments, the method may be implemented using a processor, such as processor 604. Example techniques are described herein including with respect to FIG. 14.

At 862, the techniques include receiving a signal detected from a brain of a person. The signal is detected by a transducer forming a beam in a specified direction. For example, the direction may be determined by a user, based on output from a machine learning model (e.g., described herein including with respect to FIGS. 8A, 8B, and 8E), based on prior knowledge of the direction for forming the beam, or using any other suitable techniques for determining such a direction, as embodiments of the technology are not limited in this respect.

At 864, the techniques include estimating a shift associated with the detected signal. The techniques for estimating such a shift include acts 864a and 864b, which may be performed contemporaneously, or in any suitable order.

At act 864a, statistical features associated with the detected signal are compared with statistical features associated with a previously-detected signal. In some embodiments, the techniques may include estimating a shift based on the comparison of such features. At 864b, a signal quality of the detected signal is determined. For example, the signal quality may be determined based on the statistical features of the detected signal and/or based on data (e.g., raw beam data) associated with the detected signal. In some embodiments, the output at acts 864a and 864b may be considered in conjunction with one another to determine whether an estimated shift is due to a physiological change.

The flow diagram 860 may proceed to act 866 when it is determined that the estimated shift is not due to a physiological change. At act 866, the techniques include providing an output indicative of the estimated shift. For example, the output may be used to determine an updated direction for forming a beam to correct for the shift. Additionally or alternatively, the output may be provided as feedback to a user. The user may be prompted by the feedback to correct for the shift when the hardware does not have this capability.

### Beam-Steering Interrogation Techniques

Some aspects of the technology relate to beam-steering techniques for initially identifying a region of interest. In some embodiments, a beam-steering technique informs the direction for forming the first beam (e.g., the first signal detected at 802 of flow diagram 800) and the number of beams to be formed by the transducer (e.g., a single beam, a two-dimensional plane, a sequence of two-dimensional volumes, a three-dimensional volume, etc.) at one time. In some embodiments, the beam-steering techniques may involve iterating over multiple regions of the brain (e.g., detecting and processing signals from those regions using the machine learning techniques described herein), prior to identifying the region of interest.

FIG. 9 shows example beam-steering techniques. However, it should be appreciated that any suitable beam-steering techniques may be used for identifying a region of interest, as aspects of the technology described herein are not limited in this respect.

Randomized Beam-Steering 920. In some aspects, the techniques utilize beam-steering at random directions to progressively narrow down the field-of-view to a desired target region, by exploiting a combination of various anatomical landmarks and motion in different compartments. In some embodiments, the machine learning techniques may determine the order in which the sequence is conducted. The system may instantiate a search algorithm by an initial beam (e.g., transmitting and/or receiving an initial beam) that is determined by prior knowledge, such as the relative angle and orientation of the transducer probe with respect to its position on the head. Based on the received beam data at the current and previous states, the system may determine the next best orientation and region for the next scan.

Multi-level (or multi-grid) Beam-Steering 940. In some aspects, the techniques can utilize a multi-level or multi-grid search space to narrow down the field-of-view to a desired region of interest, starting from a coarse-grained beam-steering (i.e., large spacing/angles between subsequent beams) progressively; narrowed down to a finer spacing and angle around the region of interest. The machine teaming techniques may determine the degree and area during the grid-refinement process.

Sequential Beam-Steering 960. In some aspects, the techniques can utilize a sequential beam steering in which case the device steers beams sequentially (in a specific order) over a two-dimensional plane, a sequence of two-dimensional planes positioned or oriented differently in a three-dimensional space, or a three-dimensional volume. The machine learning techniques may determine the order in which the sequence is conducted. With beam-steering merely over a two-dimensional plane or over a three-dimensional volume, the techniques may analyze a full set of beam indices/angles in two dimensions or three dimensions and determine which of the many beams scanned is a fit for the next beam. With a sequence of two-dimensional planar data and/or images (i.e., frame), the techniques may analyze consecutive frames one after another and determine the next two-dimensional plane over which the scan may be conducted.

### Data Acquisition and Processing Pipeline

As described herein, including with respect to 804 of flow diagram 800, a processor may receive, from a transducer, data indicative of a signal detected from the brain. In some embodiments, the processor may process the data according to one or more processing techniques. For example, as shown in FIG. 10, the acquired data may be processed according to pipeline 1020 for B-mode (brightness mode) imaging, CFI (color-flow imaging) and PW (pulse-wave) Doppler data. In some embodiments, any combination of processing techniques and/or any additional processing techniques may be used to process the data, as embodiments of the technology described herein are not limited in this respect.

Processing pipeline 1020 shows example processing techniques for B-mode imaging, CFI, and PW Doppler data. For each modality, raw beam data 1004 may undergo time gain compensation (TGC) 1006 to compensate for tissue attenuation. In some embodiments, the data may further undergo filtering 1008 to filter out unwanted signals and/or frequencies. In some embodiments, demodulation 1010 may be performed to remove carrier signals.

After demodulation 1010, processing techniques may vary among the different modalities. As shown, for B-mode imaging, the data may undergo envelope detection 1012 and/or logarithmic compression 1014. In some embodiments, logarithmic compression 1014 may function to adjust the dynamic range of the B-mode images. In some embodiments, the data may then undergo scan conversion 1016 for generating B-mode images. Finally, any suitable techniques 1018 may be used for post-processing the scan converted images.

For CFI, the data may undergo phase estimation 1024, which may be used to inform velocity estimation 1026. In some embodiments, after velocity estimation 1024, the data may undergo scan conversion 1016 to generate CF images. Any suitable techniques 1018 may be used for post-processing the scan converted CF images.

For PW Doppler data, the demodulated data may similarly undergo phase estimation 1024. In some embodiments, a fast Fourier transform (fft) 1028 may be applied to the output of phase estimation 1024, prior to generating sonogram 1030.

In some embodiments, any suitable data (e.g., data acquired from any point in pipeline 1020) may be used as input to machine learning techniques 1044, 1064 for determining the beam-steering strategy 1046, 1066 (e.g., the direction of beamforming for detecting the signal from the region of interest). For example, raw channel or beam data 1042 may be used as input to pipeline 1040, while B-mode and CFI data 1062 may be used as input to pipeline 1060. Other non-limiting examples of input data may include demodulated I/Q data, pre-scan conversion beam data, and scan-converted beam data.

In some embodiments, the machine learning techniques 1044, 1064 may include one or more machine learning techniques that inform the beam-steering strategy 1046, 1066. For to example, the machine learning techniques may include techniques for detecting a region of interest, localizing a region of interest, segmenting one or more anatomical structures, locking on a region of interest, correcting for movement due to shifts in hardware, correcting movement due to shifts in the beam, and/or any suitable combination of machine learning techniques. Machine learning techniques are further described herein including with respect to FIGS. 11-19.

### Detection, Localization, and Segmentation of a Region of Interest

In some embodiments, the signals detected during beam-steering, regardless of the technique, may be used to determine a current probing location from which the signals were detected. In some embodiments, the current probing location may be used to assist in detecting, locating, and/or segmenting a region of interest. The inventors have recognized that it can be challenging to determine a probing location based on observation alone, since structural landmarks in B-mode images can be subtle and easy to lose with the naked eye. Further, a full field-of-view three-dimensional space may be relatively large compared to some regions of interest. The inventors have therefore developed AI-based techniques that can be used to analyze beam data to identify the current probing location and/or guide the user and/or hardware towards the region of interest. In some embodiments, the AI-based techniques may be based on prior general structural knowledge provided in the system. For example, the AI-based techniques may exploit structural features (e.g., anatomical structures) and changes in structural features (e.g., motion) to determine a current probing position (e.g., the position of the region of the brain from which a first signal was detected).

In some embodiments the AI techniques may include using a deep neural network (DNN) framework, trained using self-supervised techniques, to predict the position of a region of interest. Self-supervised learning is a method for training computers to do tasks without labelling data. It is a subset of unsupervised learning where outputs or goals are derived by machines that label, categorize, and analyze information on their own, then draw conclusions based on connections and correlations. In some embodiments, the DNN framework may be trained to predict the relative position of two regions in the same image. For example, the DNN framework may be trained to predict the position of the region of interest with respect to an anatomical structure in a B-mode and/or CF image.

FIG. 11A shows an example diagram of the DNN framework used for estimating the relative positions of two regions in the same image. As shown, a reference patch 1102, at a given position, and a target patch 1104, at an unknown position, are used as input to an encoder 1106. The position estimator 1108 estimates the position of the target patch 1104 with respect to the position of the reference patch 1102.

In some embodiments, the DNN framework may be trained both on two-dimensional and three-dimensional images and/or four-dimensional spatiotemporal data (two- or three-dimensions for space and one-dimension for time). In some embodiments, training the DNN framework may involve obtaining a template for the region of interest. To obtain a template, a disentangling neural network may be trained to extract the region of interest structure and subject-dependent variabilities and combine them to estimate a region of interest shape for a “test” subject. FIG. 11B shows an example algorithm for template extraction. In some embodiments, to achieve a good prior template, the DNN model may be augmented with a classifier that helps the encoder identify an absolute position. This mechanism may improve upon sensitivity to a specific subject, as images from different users may be very different from one another. Additionally, the training may be augmented with a decoder that improves image quality. This may be beneficial in that the embeddings obtained from the encoder network will be rich in information for a more accurate localization.

In some embodiments, given a template of the region of interest and detected signal data (e.g., B-mode image data, CFI data, etc.) from the current probing position, the trained DNN framework may output an indication of the existence of a region of interest, a position of the region of interest with respect to the current probing position, and/or a segmentation of the region of interest. In some embodiments, the output may include a direction for forming a beam for detecting signals from the region of interest. The processor may provide instructions to the transducer to detect a signal from the region of interest by forming a beam in the determined direction.

Due to variability in size, shape, and orientation of structures in the brain (e.g., ventricles, blood vessels, brain tissue, etc.), the AI-based techniques, as described herein above, may be adapted to detect, localize, and/or segment specific structures in the brain. FIGS. 15A-17C describe example techniques for detecting, localizing, and/or segmenting example anatomical structures in the brain, according to some embodiments of the technology described herein.

Ventricle Detection, Localization, and Segmentation. In some embodiments, the techniques described herein may be used to detect, localize, and/or segment ventricles. FIG. 15A shows an example diagram 1500 of ventricles in the brain. Ventricles are critically important to the normal functioning of the central nervous system. The ventricles are four internal cavities that contain cerebrospinal fluid (CSF). CSF flows within and around the brain and spinal cord to help cushion it from injury. This circulating fluid is constantly being absorbed and replenished. There are two ventricles deep within the cerebral hemispheres called the lateral ventricles. They both connect with the third ventricle through a separate opening called the foramen of Monro. The third ventricle connects with the fourth ventricle through a long narrow tube called the aqueduct of Sylvius. From the fourth ventricle, CSF flows into the subarachnoid space where it bathes and cushions the brain. CSF is recycled (or absorbed) by special structures in the superior sagittal sinus called arachnoid villi. A balance is maintained between the amount of CSF that is absorbed and the amount that is produced. A disruption or blockage in the system can cause a build-up of CSF, which can cause enlargement of the ventricles (hydrocephalus) or cause a collection of fluid in the spinal cord (syringomyelia). Additionally, infection (such as meningitis), bleeding or blockage can change the characteristics of the CSF. Brain ventricles' shape can be very useful in diagnosing various conditions such as intraventricular hemorrhage and intracranial hypertension.

FIG. 15B shows a flow diagram 1540 of an example system for ventricle detection, localization, and segmentation. In some embodiments, the detection, localization, and segmentation algorithm may be a classical algorithm and/or a neural network 1510. In some so embodiments, the device 1502 provides data, such as B-mode image data as input to the neural network 1510. In some embodiments, additional input, such as location prior 1504, shape prior 1506, and subject information 1508, may be provided as input to the neural network. The location prior 1504 may be indicative of an expected location of the ventricle within the brain. The shape prior 1506 may be indicative of an expected shape of the ventricle. In some embodiments, the location and shape priors 1504, 1506 may be determined based on training data and/or prior knowledge. Example location and shape priors are described herein, including with respect to FIG. 15C. In some embodiments, the subject information 1508 may be used to identify subject dependent variabilities that may depend on age, sex, and/or any other suitable factors. In some embodiments, based on the input, the neural network 1510 may provide segmented results 1512 as output.

FIG. 15C shows a flow diagram illustrating an example segmentation of a ventricle. In some embodiments, data 1562 may be received from the device 1502. In some embodiments, the data may undergo one or more data processing techniques prior to segmentation. After beam-forming and envelope detection, ultrasound data consists of nf×nd×ns tensor, where of represents number of frames, nd represents number of samples in depth and, ns represents number of sensors. The depth data may contain high frequency information due to inherent speckle noise. However, the brain ventricles are relatively large regions that do not produce high frequency speckle noise. To reduce the inherent speckle noise, a Gaussian averaging may be applied. Also, slow drifts in the data may mislead the algorithm as the edges of ventricles are a band limited feature in depth data. Hence, a dc blocker may be applied to depth data as a high-pass filter. In some embodiments, the dc blocker is defined as:

$$\begin{matrix}y ( n ) = g \vert x ( n ) - x ( n - 1 ) \vert + Ry ( n - 1 ) & Equation 1 \\ g = \frac{1 + R}{2} & Equation 2\end{matrix}$$

For example, as shown in the flow diagram 1560 of FIG. 15C, the depth signal 1562 may be filtered at 1564 to generate filtered beam data. After applying the filter, a scan conversion may be performed to generate a filtered image, as shown at 1566.

In some embodiments, the segmentation techniques may be used to detect plateaus in the filtered image, while maintaining spatial compactness. An example segmentation algorithm is described by Kim et. al. (Improved simple linear iterative clustering super pixels. In 2013 IEEE ISCE, pages 259-260. IEEE, 2013.), which is incorporated herein by reference in its entirety. In some embodiments, this algorithm generates super-pixels by clustering pixels based on their color similarity and proximity in the image plane. This may be done in the five-dimensional [labxy] space, where [lab] is the pixel color vector in CIELAB color space and xy is the pixel position. An example distance measure (Equation 3) is described by Doersch et. al. (Unsupervised visual representation learning by context prediction. In Proc. IEEE International Conference on Computer Vision, pages 1422-1430, 2015.), which is incorporated herein by reference in its entirety.

$$\begin{matrix}D = {D}_{lab} + \frac{m}{s} {D}_{xy} \\ where \text{:} & Equation 3 \\ s = \sqrt{\frac{N}{K}} , and & Equation 4 \\ {D}_{lab}^{i , j} = \sqrt{{( {l}_{i} - {l}_{j} )}^{2} + {( {a}_{i} - {a}_{j} )}^{2} + {( {b}_{i} - {b}_{j} )}^{2}} , and & Equation 5 \\ {D}_{x , y}^{i , j} = \sqrt{{( {x}_{i} - {x}_{j} )}^{2} + {( {y}_{i} - {y}_{j} )}^{2}} & Equation 6\end{matrix}$$

Here, s represents an estimate of super-pixel size which may be computed as the square root ratio of N number of pixels in image and k number of super-pixels. An example of a segmented. image is shown at 1568 of flow diagram 1560.

In some embodiments, the target segment (e.g., the ventricle) may include a set of characteristics (e.g., location prior, shape prior, etc.) that may be leveraged during detection. For example, discriminating features may include (a) average pixel intensity, (b) depth, and (c) shape. To incorporate the depth prior (score), a Gaussian kernel may be formed (e.g., σ=nd/5 centered at nd/2, for an example of 5 samples), as ventricles are estimated to be positioned at about the center of the head, with a peak value normalized to one. This one-dimensional vector may then be scan converted to the ultrasound image space. Accordingly, the depth score may be computed as the average of kernel values belonging to that cluster. Flow diagram 1560 illustrates, at 1570, example depth scores (top), calculated according to the techniques described herein. As shown, clusters located near central depths in the image may have a higher score than those clusters located at shallower and/or deeper depths.

In some embodiments, pixels that belong to ventricles may have relatively lower or higher intensity than other pixels. In some embodiments, computing an intensity score for a cluster may include normalizing values to have a mean of zero and a standard deviation of one. The negative average intensity value for each cluster may be computed and transformed according to the nonlinearity in Equation 7, below:

$${score}_{i} ( x ) = \frac{1}{1 + {e}^{\frac{- ( x + 1.5 )}{0.6}}} \qquad Equation 7$$

As a result, clusters having a lower intensity may result in a higher score. Flow diagram illustrates, at 1570, example intensity scores (bottom), calculated according to the techniques described herein. As shown, clusters having a lower intensity may result in a higher intensity score.

In some embodiments, ventricles may be also viewed as a particular shape (e.g., shape prior). For example, the ventricles may be viewed as having a similar shape to that of a butterfly in a two-dimensional transcranial ultrasound image. In some embodiments, the shape may be used as a template for scale and invariant shape matching. After smoothing, the template may be used to extract a reference contour for shape scoring. In some embodiments, a contour may be represented as a set of points. For example, the contour may be represented as:

$${cntr}_{i} = [ [ {x}_{0}^{i} , {y}_{0}^{i} ] , [ {x}_{1}^{i} , {y}_{1}^{i} ] , … , [ {x}_{{n}_{i}}^{i} , {y}_{{n}_{i}}^{i} ] ] \qquad Equation 8$$

The center of the contour may be represented as:

$${O}_{i} = [ \frac{1}{{n}_{i}} {\sum }_{j = 0}^{{n}_{i}} {x}_{j}^{i} , \frac{1}{{n}_{i}} {\sum }_{j = 0}^{{n}_{i}} {y}_{j}^{i} ] \qquad Equation 9$$

In some embodiments, the contour distance curve may be formed by computing the Euclidean distance of every point in cntrᵢ to its center Oᵢ. To mitigate the scale variability, every Dᵢ may be normalized to m_{D}_{ᵢ}=0 and σ_{D}_{ᵢ}=1, ∀i, then a spline may be fit, and all curves may be resampled (e.g., to 200). In addition, to mitigate the rotation variability and to compute a score, the cross-correlation of the template and contours at lags may be estimated. This may be repeated for the first, second and third order derivative of template and other contours and the average of maximum cross-correlation is reported as score. Note that the lag corresponding to maximum correlation may he used to estimate the angle of rotation. The final score for each cluster may then be computed by applying the following nonlinearity:

$${score}_{s} ( x ) = \frac{1}{1 + {e}^{- 10 x}} \qquad Equation 10$$

Flow diagram 1560 illustrates, at 570, example shape scores (middle) for each of the clusters. In some embodiments, clusters that have a shape that resemble the shape prior (e.g., the butterfly) may result in a higher shape score.

In some embodiments, a final score may be computed for each cluster by computing the product of the depth, shape, and intensity scores. Example final scores are shown at 1572 of flow diagram 1560. The final selection may be performed by selecting an optimal (e.g., maximum, minimum, etc.) score that satisfies a threshold. For example, selecting a maximum score that exceeds a threshold of 0.75. An example final selection of a cluster is shown at 1574 of flowchart 1560. As shown, the selected cluster corresponds to the highest score from among the scores associated with clusters at 1572.

Circle of Willis Detection and Segmentation. In some embodiments, the techniques described herein may be used to detect, localize, and segment the circle of Willis. FIG. 16A shows an example diagram 1600 of the circle of Willis. The circle of Willis is a collection of arteries at the base of the brain. The circle of Willis provides the blood supply to the brain. It connects two arterial sources together to form an arterial circle, which then supplies oxygenated blood to over 80% of the cerebrum. The structure encircles the middle region of the brain, including the stalk of the pituitary gland and other important structures. The two carotid arteries supply blood to the brain through the neck and lead directly to the circle of Willis. Each carotid artery branches into an internal and external carotid artery. The internal carotid artery then branches into the cerebral arteries. This structure allows all of the blood from the two internal carotid arteries to pass through the circle of Willis. The internal carotid arteries branch off from here into smaller arteries, which deliver much of the brain's blood supply. The structure of the circle of Willis includes, left and right middle cerebral arteries (MCA), left and right internal carotid arteries (ICA), left and right anterior cerebral arteries (ACA), left and right posterior cerebral arteries (PCA), left and right posterior communicating arteries, basilar artery, anterior communicating artery.

As opposed to ventricle detection, localization, and segmentation, template matching methods may not be feasible for the circle of Willis, due to the large template that would be needed. Template matching methods may not work well for large templates because the speckle noise in the image may mislead the algorithm. Therefore, the circle of Willis may be detected, localized, and/or segmented using the methods described herein including at least with respect to FIGS. 16A-17C. A first example method may include separately detecting, localizing, and segmenting different regions of the circle of Willis according to template matching techniques (e.g., such as the techniques described herein, including with respect to FIGS. 15B-C), as shown in flow diagram 1650 of FIG. 16B. As shown, data, such as B-mode image data, from the device 1652 may be processed to detect, localize, and segment different regions 1654 of the circle of Willis. For example, the different regions may include the left and right MCA, left and right ICA, left and right PCA, and left and right ACA. In some embodiments, the techniques may include processing the data with a neural network (e.g., neural network 1510) to separately detect, localize, and segment each region. In some embodiments, the segmented regions may then be fused 1656 and provided as output 1658.

Additionally or alternatively, a second example method for detecting, localizing, and segmenting the circle of Willis may include applying techniques described herein for detecting, localizing, and segmenting blood vessels. In some embodiments, as described herein, including at least with respect to FIGS. 17A-C, shape priors and neural networks may be used to extract the circle of Willis from B-mode and CF-images.

Vessel Diameter and Blood Volume Rate. In some embodiments, techniques may be used to determine a vessel diameter and blood volume rate. The inventors have recognized that traditional matching methods used in computer vision are vulnerable to error in presence of slight shape changes, rotation, and scale. As a result, it may be challenging to determine such blood vessel metrics. The inventors have therefore developed techniques for finding (e.g., detecting and/or localizing) a vessel from B-mode and CF images based on template matching, while addressing these issues.

FIG. 17A shows a flow diagram 1700 of an example system for determining blood vessel diameter and curve. As shown, device 1702 may provide data, such a B-mode image and CF image data, as input to the system. In some embodiments, the techniques utilize pre-trained neural network models and use the output of the middle layers to perform scale and rotation invariant feature extraction. The features may be compared to the features extracted from a template of a vessel to indicate the region of interest location (e.g., vessel localization at 1704 of flow diagram 1700). This may help to create a region of B-mode and color-flow image frames such that vessel location variations are minimal.

As a result, the techniques may obtain a set of frames from the region of interest that are well aligned even in the face of heartbeat, respiration, and probe-induced movements. In some embodiments, image enhancement techniques 1706 may be applied to the aligned region of interest. In some embodiments, averaging the frames may reduce the noise and result in good contrast between the vessel and background. Next, a two-component mixture of Gaussians may be used to cluster foreground and background pixels together. For example, the two components may include pixel value and pixel position. In some embodiments, a polynomial curve may be fit to the foreground and a mask may be created by drawing vertical lines of length r, centered at polynomial. To obtain the best fit, a parameter search 1708 may be conducted over polynomial order and r 1710. This may result in an analytical equation for vessel shape and vessel radius, output at 1712. In some embodiments, vessel shape discovery may also be useful in determining the beam angle to the blood-flow direction that improves PW measurement and accordingly the cerebral blood flow velocity estimates.

FIG. 17B shows an example of determining the diameter and curve of a blood vessel. As shown, the blood vessel is localized at 1742, indicated by the highlighted vessel and border outlining the highlighted vessel. At 1744, alignment and enhancement techniques are applied to the region of interest to reduce the noise and improve the contrast between the vessel and the background. At 1746, a polynomial curve is fit, and a parameter search is conducted to determine output 1748, which may include diameter and curve of the vessel.

As described above, these techniques may be used to detect, localize, and/or segment the circle of Willis. For example, FIG. 17C shows a segmentation of the middle cerebral artery, along with a vessel diameter estimation,

### Active Guidance Mechanism for Locking, Sensing, and Tracking

In some embodiments, the detection and localization techniques described herein may help to determine an approximate position of a region of interest. However, due to variabilities among subjects (e.g., among the brains of subjects), there may be slight inaccuracies associated with the estimated position of the region of interest. In some embodiments, it may be desirable to address these inaccuracies and precisely lock onto the region of interest for an individual. In some embodiments, a fine-tuning mechanism may be deployed in a closed-loop system to precisely lock onto the region of interest. In some embodiments, the techniques may include analyzing one or more signals detected by the transducer to determine an updated direction for forming a beam for precisely detecting signals from the region of interest.

FIG. 12 is a block diagram 1200 showing a system for locking onto a region of interest, according to some embodiments of the technology described herein. In some embodiments, device 1202 may detect signals from the brain. The data may be used to generate one or more B-mode and/or CF image frames 1204. The image frames 1204, along with template 1206, may be used as input to an algorithm for detection and localization 1208 of the region of interest to determine one or more scores (e.g., the scores described with respect to FIG. 15B), a predicted .m position of the region of interest, and/or a direction for forming the beam for detecting signals from a region of interest. In some embodiments, a reinforcement-learning based algorithm 1210 may map the output of the detection and localization algorithm 1208, the image frame(s) 1204, and the template 1206 to a set of sparse pulse-wave (PW) beams to explore the proximity of the region of interest. In some embodiments, the reinforcement-learning based algorithm 1210 may analyze the quality of the signal 1212, such as the signal-to-noise ratio (SNR), to determine a candidate region of interest. For example, the reflected power of Doppler may be used to estimate the SNR to determine a candidate region of interest. The processor may provide the output of the reinforcement-learning based algorithm 1210 to the transducer, instructing the transducer to detect the signal from the refined position (e.g., the candidate region of interest). In some embodiments, the process is repeated (e.g., using beam data acquired from the candidate region of the brain) until the algorithm converges and/or a time threshold is reached. In some embodiments, detecting the signal from the refined position may help to lock on the region of interest and improve the SNR (e.g., increase the SNR).

Active target tracking. The inventors have recognized that, during continuous recording, it can be challenging to keep the hardware on target, despite the closed-loop mechanisms for locking onto the region of interest. Shifts and/or drifts in the hardware (e.g., the transducer) may occur, even though the hardware may be designed to lock in place (e.g., on the region of interest) and keep a sturdy hold in position. In some embodiments, a live tracking system may be used to address hardware shifts and/or drifts based on a Kaltman filter.

FIG. 13 is a block diagram 1300 showing a system for determining and/or measuring drifts associated with hardware. In some embodiments, the techniques include acquiring PW beams 1304, in PW Doppler mode, from one or a few angles in high frequency using device 1302. The techniques may further include recording a B-mode image 1306, 1310 at a relatively low frequency (e.g., once every second and/or every few seconds) using device 1302, during the PW recordings. In some embodiments, using a speckle correlation analysis, a most recently recorded B-mode image frame 1306 may be used as a reference to indicate the location and possible angular shifts in the current PW beam 1304. An estimated undesirable shift 1308 is provided as input to a motion prediction and compensation framework (e.g., a Kalman filter) 1314, which determines an updated direction for forming a beam (e.g., a PW beam) to keep the beam on target (e.g., on the region of interest). In some embodiments, the updated direction may be provided as feedback 1316 to the transducer for course-correction. Additionally or alternatively, to ensure that the reference B-mode image frame 1306 still captures the correct slice and to determine the template shift 1312 over time, the B-mode image frame 1306 is compared to a previously-acquired B-mode image frame 1310. For example, the B-mode image frames 1306, 1310 may be compared using an order-one Markovian model. In some embodiments, the output of the comparison may be provided as feedback 1316 to the transducer to adjust for B-mode frame shifts (e.g., update the direction for forming the beam). Additionally or alternatively, the output of the comparison may be provided as feedback 1316 to a user if the device focus point is moving out of the plane of the target region and there is no hardware capability for correcting for the shift.

Course Correcting Component. The inventors have further recognized that, though the techniques may lock the system on target, the beam may gradually shift, or the contact quality may change during the course of measurement. To address this, in some embodiments, the techniques may monitor the signal quality and, upon observing a statistical shift that does not translate to physiological changes, it may (a) perform a limited search around the region of interest to fix the limited shift without interrupting the measurements, and/or (b) upon observing substantial dislocations, engages the reinforcement-learning algorithm for realigning and/or alerting the user of contact issues if the search was unsuccessful.

FIG. 14 is a block diagram 1400 showing a system for determining and/or measuring shifts in the beam. As shown, the techniques include acquiring a beam in PW Doppler mode at a time t₁, using device 1402. In some embodiments, the system extracts the statistical features 1406 of the beam acquired at time t₁. The statistical features 1406, along with the raw beam data 1404 are used as input to a signal quality estimator 1412 to determine if the data satisfies certain conditions (e.g., the signal quality is satisfactory). Additionally or alternatively, the statistical features 1406 of the beam 1404 acquired at time t₁ are compared to statistical features extracted from a previously-acquired beam 1408 to estimate statistical shifts 1410. In some embodiments, the statistical shift estimator 1410 may include a Siamese DNN, which may look for a substantial shift, as well as slow drifts, in statistics of the signal and classify the nature of the shifts and/or drifts. In some embodiments, the outputs of the statistical shift estimator 1410 and the signal quality estimator 1412 may be used to determine a course of action if a shift occurs (e.g., using predictor 1414). For example, the output may be provided to a DNN-based Kalman filter for tracking three-dimensional motion using the signal quality. In some embodiments, the output of the predictor 1414 may be provided as feedback 1416 to the transducer for forming a beam in an updated direction (e.g., for correcting for the shift). Additionally or alternatively, feedback 1416 may be provided to a user for adjusting the hardware and/or providing an indication of the shift.

Autonomous Sensing

In some embodiments, once locked onto a region of interest, the system (e.g., system 600) may continuously and/or autonomously monitor the region of interest using any suitable ultrasound modality. For example, ultrasound modalities may include continuous wave (CW) Doppler, pulse wave (PW) Doppler, pulsatile-mode (P-mode), pulse-wave-velocity (PWV), color flow imaging (CFI), power Doppler (PD), motion mode (M-mode), and/or any other suitable ultrasound modality, as aspects of the technology described herein are not limited in that respect.

Additionally or alternatively, once locked onto a region of interest, the system (e.g., system 600) may sense and/or monitor brain metrics from the region of interest. For example, brain metrics may include intracranial pressure (ICP), cerebral blood flow (CBF), cerebral perfusion pressure (CPP), intracranial elastance (ICE), and/or any suitable brain metric, as aspects of the technology described herein are not limited in this respect.

### Artificial Intelligence (AI) in Smart Beam Steering

As described herein, AI can be used on various levels such as in guiding beam steering and beam forming, detection, localization, and segmentation of different landmarks, tissue types, vasculature and physiological abnormalities, detection and localization of blood flow and motion, autonomous segmentation of different tissue types and vasculature, autonomous ultrasound sensing modalities, and/or sensing and monitoring brain metrics, such as intracranial pressure, intracranial elastance, cerebral blood flow, and/or cerebral profusion.

In some embodiments, beam-steering may employ one or more machine learning algorithms in the form of a classification or regression algorithm, which may include one or more sub-components such as convolutional neural networks, recurrent neural networks such as LSTMs and GRUs, linear SVMs, radial basis function SVMs, logistic regression, and various techniques from unsupervised learning such as variational autoencoders (VAE), generative adversarial networks (GANs) which are used to extract relevant features from the raw input data.

Exemplary steps 1800 often undertaken to construct and deploy the algorithms described herein are shown in FIG. 18, including data acquisition, data preprocessing, building a model, training the model, evaluating the model, testing, and adjusting model parameters.

FIG. 19 shows a convolutional neural network 1900 that may be employed by the AEG-device. The statistical or machine learning model described herein may include the convolutional neural network 1900, and additionally or alternatively another type of network, suitable for predicting frequency, amplitude, acoustic beam profile, and other requirements, such as expected temperature elevation and/or radiation force, etc. As shown, the convolutional neural network comprises an input layer 1904 configured to receive information about the input 1902 (e.g., a tensor), an output layer 1908 configured to provide the output (e.g., classifications in an n-dimensional representation space), and a plurality of hidden layers 1906 connected between the input layer 1904 and the output layer 1908. The plurality of hidden layers 1906 include convolution and pooling layers 1910 and fully connected layers 1912.

The input layer 1904 may be followed by one or more convolution and pooling layers 1910. A convolutional layer may comprise a set of filters that are spatially smaller (e.g., have a smaller width and/or height) than the input to the convolutional layer (e.g., the input 1902). Each of the filters may be convolved with the input to the convolutional layer to produce an activation map (e.g., a 2-dimensional activation map) indicative of the responses of that filter at every spatial position. The convolutional layer may be followed by a pooling layer that down-samples the output of a convolutional layer to reduce its dimensions. The pooling layer may use any of a variety of pooling techniques such as max pooling and/or global average pooling. In some embodiments, the down-sampling may be performed by the convolution layer itself (e.g., without a pooling layer) using striding.

The convolution and pooling layers 1910 may be followed by fully connected layers 1912. The fully connected layers 1912 may comprise one or more layers each with one or more neurons that receives an input from a previous layer (e.g., a convolutional or pooling layer) and provides an output to a subsequent layer (e.g., the output layer 1908). The fully connected layers 1912 may be described as “dense” because each of the neurons in a given layer may receive an input from each neuron in a previous layer and provide an output to each neuron in a subsequent layer. The fully connected layers 1912 may be followed by an output layer 1908 that provides the output of the convolutional neural network. The output may be, for example, an indication of which class, from a set of classes, the input 1902 (or any portion of the input 1902) belongs to. The convolutional neural network may be trained using a stochastic gradient descent type algorithm or another suitable algorithm. The convolutional neural network may continue to be trained until the accuracy on a validation set (e.g., a held-out portion from the training data) saturates or using any other suitable criterion or criteria.

It should be appreciated that the convolutional neural network shown in FIG. 19 is only one example implementation and that other implementations may be employed. For example, one or more layers may be added to or removed from the convolutional neural network shown in FIG. 19. Additional example layers that may be added to the convolutional neural network include: a pad layer, a concatenate layer, and an upscale layer. An upscale layer may be configured to up-sample the input to the layer. An ReLU layer may be configured to apply a rectifier (sometimes referred to as a ramp function) as a transfer function to the input. A pad layer may be configured to change the size of the input to the layer by padding one or more dimensions of the input. A concatenate layer may be configured to combine multiple inputs (e.g., combine inputs from multiple layers) into a single output. As another example, in some embodiments, one or more convolutional, transpose convolutional, pooling, un-pooling layers, and/or batch normalization may be included in the convolutional neural network. As yet another example, the architecture may include one or more layers to perform a nonlinear transformation between pairs of adjacent layers. The non-linear transformation may be a rectified linear unit (ReLU) transformation, a sigmoid, and/or any other suitable type of non-linear transformation, as aspects of the technology described herein are not limited in this respect.

Convolutional neural networks may be employed to perform any of a variety of functions described herein. It should be appreciated that more than one convolutional neural network may be employed to make predictions in some embodiments. Any suitable optimization technique may be used for estimating neural network parameters from training data. For example, one or more of the following optimization techniques may be used: stochastic gradient descent (SGD), mini-batch gradient descent, momentum SGD, Nesterov accelerated gradient. Adagrad, Adadelta, RMSprop, Adaptive Moment Estimation (Adam), AdaMax, Nesterov-accelerated Adaptive Moment Estimation (Nadam), AMSGrad.

### Example Computer Architecture

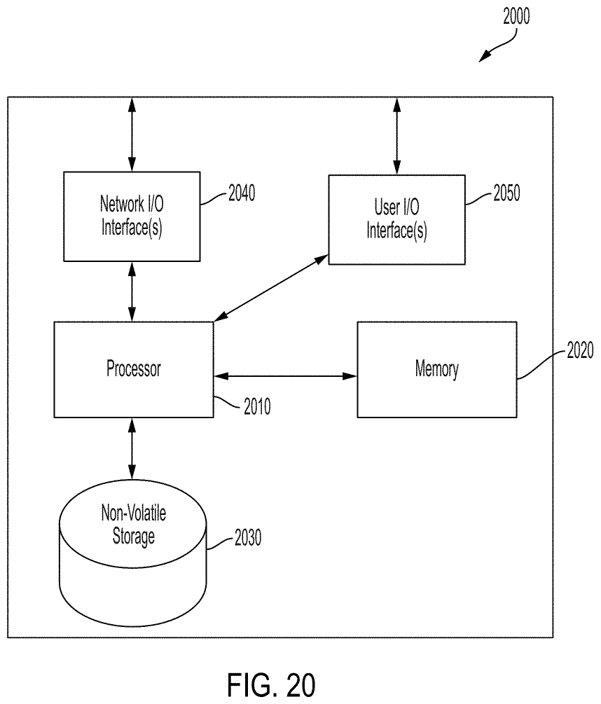

An illustrative implementation of a computer system 2000 that may be used in connection with any of the embodiments of the technology described herein is shown in FIG. 20. The computer system 2000 includes one or more processors 2010 and one or more articles of manufacture that comprise non-transitory computer-readable storage media (e.g., memory 2020 and one or more non-volatile storage media 2030). The processor 2010 may control writing data to and reading data from the memory 2020 and the non-volatile storage device 2030 in any suitable manner, as the aspects of the technology described herein are not limited in this respect. To perform any of the functionality described herein, the processor 2010 may execute one or more processor-executable instructions stored in one or more non-transitory computer-readable storage media (e.g., the memory 2020), which may serve as non-transitory computer-readable storage media storing processor-executable instructions for execution by the processor 2010.

Computing device 2000 may also include a network input/output (I/O) interface 2040 via which the computing device may communicate with other computing devices (e.g., over a network), and may also include one or more user I/O interfaces 2050, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

The embodiments described herein can be implemented in any of numerous ways. For example, the embodiments may be implemented using hardware, software, or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described herein can be generically considered as one or more controllers that control the functions discussed herein.

The one or more controllers can be implemented in numerous ways, such as with dedicated hardware, or with general purpose hardware (e.g., one or more processors) that is programmed using microcode or software to perform the functions recited herein.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the functions discussed herein of one or more embodiments. The computer-readable medium may be transportable such that the program stored thereon can be loaded onto any computing device to implement aspects of the techniques discussed herein. In addition, it should be appreciated that the reference to a computer program which, when executed, performs any of the functions discussed herein, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques discussed herein.

The terms “program” or “software” are used herein in a generic sense to refer to any type of computer code or set of processor-executable instructions that can be employed to program a computer or other processor to implement various aspects of embodiments as discussed herein. Additionally, it should be appreciated that according to one aspect, one or more computer programs that when executed perform methods of the disclosure provided herein need not reside on a single computer or processor but may be distributed in a modular fashion among different computers or processors to implement various aspects of the disclosure provided herein.

Processor-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in one or more non-transitory computer-readable storage media in any suitable form. For simplicity of illustration, data structures may be shown to have fields that are related through location in the data structure. Such relationships may likewise be achieved by assigning storage for the fields with locations in a non-transitory computer-readable medium that convey relationship between the fields. However, any suitable mechanism may be used to establish relationships among information in fields of a data structure, including through the use of pointers, tags or other mechanisms that establish relationships among data elements.

Also, various inventive concepts may be embodied as one or more processes, of which examples have been provided. The acts performed as part of each process may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, and/or ordinary meanings of the defined terms.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

The phraseology and terminology used herein is for the purpose of description and should not be regarded as limiting. The use of “including,” “comprising:” “having,” “containing,” “involving,” and variations thereof, is meant to encompass the items listed thereafter and additional items.

Having described several embodiments of the techniques described herein in detail, various modifications, and improvements will readily occur to those skilled in the art. Such modifications and improvements are intended to be within the spirit and scope of the disclosure. Accordingly, the foregoing description is by way of example only, and is not intended as limiting. The techniques are limited only as defined by the following claims and the equivalents thereto.

While some aspects and/or embodiments described herein are described with respect to certain brain conditions, these aspects and/or embodiments may be equally applicable to monitoring and/or treating symptoms for any suitable neurological disorder or brain condition. Any limitations of the embodiments described herein are limitations only of those embodiments and are not limitations of any other embodiments described herein.

What is claimed is:

1. A method, comprising:
forming a beam in a direction relative to a brain of a person, the direction being determined by a machine learning model trained on data from prior signals detected from a brain of one or more persons; and
after forming the beam, detecting a signal from a region of interest of the brain of the person.

2. The method as claimed in claim 1, further comprising:
detecting a first signal from a first region of the brain of the person; and
providing data from the first signal as input to the machine learning model to obtain an output indicative of the direction for forming the beam.

3. The method as claimed in claim 1, further comprising:
prior to detecting the signal from the region of interest, forming a first beam in a first direction relative to the brain of the person to detect a first signal, wherein the first direction is determined based on an angle and/or an orientation of the transducer with respect to exterior anatomy of the person.

4. The method as claimed in claim 1, further comprising:
detecting a first plurality of signals from a first region of the brain of the person; and
providing data from the first plurality of signals as input to the machine learning model to obtain an output indicative of the direction for forming the beam.

5. The method as claimed in claim 1, wherein the signal is one of a plurality of signals detected from the region of interest of the brain, the method further comprising:
forming a plurality of beams, wherein adjacent beams of the plurality of beams are separated by an angle determined by the machine learning model; and
after forming the plurality of beams, detecting the plurality of signals from the region of interest of the brain.

6. The method as claimed in claim 5, further comprising:
forming a first plurality of beams, wherein adjacent beams of the first plurality of beams are separated by a first angle;
after forming the first plurality of beams, detecting a first plurality of signals from the brain of the person; and
providing data from the first plurality of signals as input to a machine learning model to obtain an output indicative of the angle by which the adjacent beams of the plurality of beams are separated.

7. The method as claimed in claim 6, wherein the angle determined by the machine learning model is narrower than the first angle.

8. The method as claimed in claim 6, wherein the machine learning model is configured to determine the direction for forming the beam based on a direction of at least one of the first plurality of beams relative to the brain of the person.

9. The method as claimed in claim 1, wherein the signal is one of a plurality of signals, the method further comprising:
forming a plurality of beams over a two-dimensional plane determined by the machine learning model; and
after forming the plurality of beams, detecting the plurality of signals from the region of interest of the brain.

10. The method as claimed in claim 9, further comprising:
forming a first plurality of beams over a first two-dimensional plane to detect a first plurality of signals from a first region of the brain of the person; and
providing data from the first plurality of signals as input to the machine learning model to obtain an output indicative of the two-dimensional plane over which to form the plurality of beams.

11. The method as claimed in claim 2, wherein the machine learning model is configured to:
determine a predicted position of the region of interest of the brain based on the provided data; and
based on the predicted position, determine the direction for forming the beam.

12. The method as claimed in claim 2, wherein the provided data is indicative of motion and/or additional information of one or more structures in the brain, and wherein the provided data comprises brightness mode (B-mode) image data, color-flow image (CFI) data, and/or raw beam data.

13. The method as claimed in claim 11, wherein the machine learning model is further configured to determine the predicted position of the region of interest based on a template of the region of interest.

14. The method as claimed in claim 11, wherein the machine learning model is further configured to:
determine, based on the provided data, a predicted position of the first region of the brain from which the first signal was detected; and
determine the predicted position of the region of interest of the brain with respect to the predicted position of the first region of the brain.

15. The method as claimed in claim 1, wherein the signal is one of a plurality of signals detected from the region of interest of the brain, the method further comprising:
forming a plurality of beams over a two-dimensional plane, over a sequence of two-dimensional planes, and/or over a three-dimensional volume determined by the machine learning model; and
after forming the plurality of beams, detecting the plurality of signals from the region of interest of the brain.

16. The method as claimed in claim 1, wherein the machine learning model is configured to estimate a shape of the region of interest for the person based on a subject-dependent variable.

17. The method as claimed in claim 1, further comprising determining an existence, location, and/or segmentation of a feature in the brain, the feature comprising blow flood, motion, an anatomical structure, and/or an anatomical abnormality.

18. The method as claimed in claim 17, wherein the anatomical structure includes one or more of ventricles and vasculature, and wherein the anatomical abnormality includes one or more of focal seizures, hemorrhage, bleed, tumor, stroke, and emboli.

19. The method as claimed in claim 1, further comprising processing the detected signal to based on an ultrasound sensing technique, the ultrasound sensing technique including one or more of brightness mode (B-mode), continuous wave (CW) Doppler, pulse wave (PW) Doppler, pulsatile-mode (P-mode), pulse-wave-velocity (PAW), color-flow imaging (CFI), power Doppler (PD), and motion mode (M-mode).

20. The method as claimed in claim 1, further comprising determining a brain metric based on the detected signal, wherein the brain metric includes one or more of intracranial pressure (ICP), cerebral blood flow (CBF), cerebral perfusion pressure (CPP), and intracranial elastance (ICE).
